SEQUENCE LISTING (9 sequences)

SEQ ID NO: 1 - protein, 5 aa, Mus musculus
MATVA

SEQ ID NO: 2 - DNA, 49 bp, Mus musculus
cccgcctgccggaagtcggctgcctgaccatggcgacagtggcggagca

SEQ ID NO: 3 - DNA, 49 bp, Mus musculus; identical to SEQ ID NO: 2

SEQ ID NO: 4 - DNA, 19 bp, Artificial Sequence
  gRNA1
tgcctgaccatggcgacag

SEQ ID NO: 5 - DNA, 20 bp, Artificial Sequence
  gRNA2
ggaagtcggctgcctgacca

SEQ ID NO: 6 - DNA, 49 bp, Artificial Sequence; identical to SEQ ID NO: 2
  Sptcl1-/- gRNA1

SEQ ID NO: 7 - DNA, 49 bp, Artificial Sequence; identical to SEQ ID NO: 2
  Sptcl1+/- gRNA2

SEQ ID NO: 8 - DNA, 49 bp, Artificial Sequence; identical to SEQ ID NO: 2
  Sptcl1+/- gRNA2

SEQ ID NO: 9 - DNA, 49 bp, Artificial Sequence; identical to SEQ ID NO: 2
  Sptcl1+/- gRNA2

(19) United States
(12) Patent Application Publication
(10) Pub. No.: US 2022/0288004 A1
(43) Pub. Date: Sep. 15, 2022

(54) TREATMENT AND PREVENTION OF AGING RELATED-DISEASE AND/OR AGING BY THE INHIBITION OF SPHINGOLIPIDS

(71) Applicant: ECOLE POLYTECHNIQUE FEDERALE DE LAUSANNE (EPFL), Lausanne (CH)

(72) Inventors: Pirkka-Pekka LAURILA, Renens (CH); Johan AUWERX, Buchillon (CH)

(21) Appl. No.: 17/762,325

(22) PCT Filed: Sep. 23, 2020
(86) PCT No.: PCT/EP2020/076459

§ 371 Date: Mar. 21, 2022

(30) Foreign Application Priority Data
Sep. 23, 2019 (EP) 19198977.1

(51) Int. Cl. A61K 31/201; A61K 31/7105; A61K 31/713; C12N 9/04; C12N 9/02; C12N 9/10; C12N 9/12; C12N 9/16; C12N 9/78

(52) U.S. Cl. CPC A61K 31/201; A61K 31/7105; A61K 31/713; C12N 9/0006; C12N 9/0071; C12N 9/1029; C12N 9/1205; C12N 9/1288; C12N 9/16; C12N 9/78; C12Y 101/01102; C12Y 114/19; C12Y 203/01024; C12Y 207/01091; C12Y 207/08027; C12Y 301/03023; C12Y 301/04012; C12Y 305/01023

(57) ABSTRACT

The present invention relates to an inhibitor of sphingolipids for use in the treatment or prevention of an aging related-disease and/or aging.

The present invention relates to an inhibitor of sphingolipids for use in the treatment or prevention of an aging related-disease and/or aging.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

The human population is getting older. Worldwide, the average life expectancy at birth was 71 years (70 years for males and 72 years for females) over the period 2010-2015 according to United Nations World Population Prospects 2015 Revision. The number and proportion of older people in society is therefore constantly growing. Currently, over 11% of the world's current population are people aged 60 and older.

Ageing is among the greatest known risk factors for most diseases. Of the roughly 150,000 humans who die each day worldwide, about two thirds die from age-related causes. Some strategies for the prevention and delay of aging are known. Non-limiting examples are caloric restriction, not too much and not too little sleep (between 5 and 7 hours), physical exercise and the avoidance of stress. However, there is still an ongoing need for new strategies to treat and prevent aging related-diseases and aging in general. This need is addressed by the present invention.

Accordingly, the present invention relates to an inhibitor of sphingolipids for use in the treatment or prevention of an aging related-disease and/or aging.

The present invention likewise relates to a method for treating or preventing an aging related-disease and/or aging in a subject in need thereof comprising administering to the subject a therapeutically effective amount of an inhibitor of sphingolipids.

In this connection it is preferred that treatment or prevention of an aging related-disease and/or aging is at least in part achieved by activating the moygenic differentiation in muscle progenitor cells.

In this connection it also preferred that the treatment or prevention of an aging related-disease and/or aging results in improved fitness of the subject to be treated. As means for determining improved fitness an ergometer test or a lactate test may be used before vs. after/during the treatment.

The inhibitor preferably specifically inhibits sphingolipids, more preferably specifically inhibits the de novo synthesis of sphingolipids in the subject to be treated. Specific binding designates that the inhibitor does not or essentially does not inhibit other targets, e.g. other lipids or proteins or peptides. The nature of the inhibitor of sphingolipids is not particularly limited and concrete examples of suitable inhibitors will be provided herein below. In this connection “essentially” means with increasing preference less than 10%, less than 5% and less than 1% off target inhibition as compared to the target inhibition set to 100%, when inhibition is measured under equal conditions.

The subject to be treated is preferably a mammal and most preferably a human.

Sphingolipids are a class of lipids containing a backbone of sphingoid bases, a set of aliphatic amino alcohols that includes sphingosine. Sphingosine (2-amino-4-trans-octadecene-1,3-diol) is an 18-carbon amino alcohol with an unsaturated hydrocarbon chain. Sphingolipids play important roles in signal transduction and cell recognition.

Aging represents the accumulation of changes in a subject over life time, encompassing in particular physical and psychological changes, such as a reduction of muscle strength, mobility, stamina and/or capacity of remembering. These changes may result in aging related-diseases (also designated aging-associated diseases).

An aging related-disease is a disease that is most often seen with increasing frequency with increasing senescence. Essentially, aging-associated diseases are complications arising from senescence. Aging related-disease diseases are to be distinguished from the aging process itself because all subjects become older but not all old subjects experience an aging related-disease. Examples of aging-associated diseases will provided herein below.

The inhibitors as described herein may be formulated as vesicles, such as liposomes or exosomes, or as lipid nanospheres. Liposomes have attracted great interest because of their specificity and the duration of action they offer from the standpoint of drug delivery. Liposomal cell-type delivery systems have been used to effectively deliver nucleic acids, such as siRNA in vivo into cells (Zimmermann et al. (2006) Nature, 441:111-114). Liposomes are unilamellar or multilamellar vesicles which have a membrane formed from a lipophilic material and an aqueous interior. The aqueous portion contains the composition to be delivered. Cationic liposomes possess the advantage of being able to fuse to the cell wall. Non-cationic liposomes, although not able to fuse as efficiently with the cell wall, are phagocytosed by macrophages and other cells in vivo. Exosomes are lipid packages which can carry a variety of different molecules including RNA (Alexander et al. (2015), Nat Commun; 6:7321). The exosomes including the molecules comprised therein can be taken up by recipient cells. Hence, exosomes are important mediators of intercellular communication and regulators of the cellular niche. Exosomes are useful for diagnostic and therapeutic purposes, since they can be used as delivery vehicles, e.g. for contrast agents or drugs. Lipid nanospheres suitable to be used herein are, for example, described in US 2012/040007 and WO 2004/039351.

The inhibitors can be administered to the subject at a suitable dose and/or a therapeutically effective amount.

The length of treatment needed to observe changes and the interval following treatment for responses to occur vary depending on the desired effect. The particular amounts may be determined by conventional tests which are well known to the person skilled in the art. Suitable tests are, for example, described in Tamhane and Logan (2002), “Multiple Test Procedures for Identifying the Minimum Effective and Maximum Safe Doses of a Drug”, Journal of the American statistical association, 97(457):1-9.

The inhibitors can be admixed with a pharmaceutically acceptable carrier or excipient to form a pharmaceutical composition. In accordance with the present invention, the term “pharmaceutical composition” relates to a composition for administration to a subject, preferably a human subject. The pharmaceutical composition of the invention comprises the compound(s) recited above. It may, optionally, comprise further molecules capable of altering the characteristics of the inhibitor thereby, for example, stabilizing, modulating and/or activating its function. The composition may be in solid, liquid or gaseous form and may be, inter alia, in the form of (a) powder(s), (a) tablet(s), (a) solution(s) or (an) aerosol(s). The pharmaceutical composition of the present invention may, optionally and additionally, comprise a pharmaceutically acceptable carrier. Examples of suitable pharmaceutical carriers are well known in the art and include phosphate buffered saline solutions, water, emulsions, such as oil/water emulsions, various types of wetting agents, sterile solutions, organic solvents including DMSO etc. Compositions comprising such carriers can be formulated by well known conventional methods. These pharmaceutical compositions can be administered to the subject at a suitable dose. The dosage regimen will be determined by the attending physician and clinical factors. As is well known in the medical arts, dosages for any one subject depends upon many factors, including the patient's size, body surface area, age, the particular compound to be administered, sex, time and route of administration, general health, and other drugs being administered concurrently. The therapeutically effective amount for a given situation will readily be determined by routine experimentation and is within the skills and judgement of the clinician or physician. Generally, the regimen as a regular administration of the pharmaceutical composition should be in the range of 1 μg to 5 g units per day. However, a more preferred dosage might be in the range of 0.01 mg to 100 mg, even more preferably 0.01 mg to 50 mg and most preferably 0.01 mg to 10 mg per day. Furthermore, if for example said compound is an iRNA agent, such as an siRNA, the total pharmaceutically effective amount of pharmaceutical composition administered will typically be less than about 75 mg per kg of body weight, such as for example less than about 70, 60, 50, 40, 30, 20, 10, 5, 2, 1, 0.5, 0.1, 0.05, 0.01, 0.005, 0.001, or 0.0005 mg per kg of body weight. More preferably, the amount will be less than 2000 nmol of iRNA agent (e.g., about 4.4×1016 copies) per kg of body weight, such as for example less than 1500, 750, 300, 150, 75, 15, 7.5, 1.5, 0.75, 0.15, 0.075, 0.015, 0.0075, 0.0015, 0.00075 or 0.00015 nmol of iRNA agent per kg of body weight. The length of treatment needed to observe changes and the interval following treatment for responses to occur vary depending on the desired effect. The particular amounts may be determined by conventional tests which are well known to the person skilled in the art.

The data in the appended examples herein below establish the relationship between sphingolipid metabolism and aging related-disease and aging. Sphingolipids have been associated with metabolic disease, cancer, and cardiovascular disease. The data provides evidence that sphingolipids have an independent effect on muscle regeneration, noting that aging related-diseases and aging are generally associated with loss of muscles strength. This is validated by myriocin-induced proliferation and differentiation of freshly isolated muscle stem cells (MuSCs) as well as mouse and human immortalized myoblast cell lines, demonstrating the cell-autonomous effect of sphingolipid synthesis inhibition on myogenesis. The specificity of these effects to sphingolipid de novo synthesis pathway, and not to off-target effects of myriocin, is verified by myogenesis-promoting effects of genetic inactivation of different members of the pathway, including Sptlc1 and Cers2, the most abundant ceramide synthase in skeletal muscle. These findings indicate that inhibition of sphingolipid de novo synthesis pathway is expected to attenuate frailty and sarcopenia.

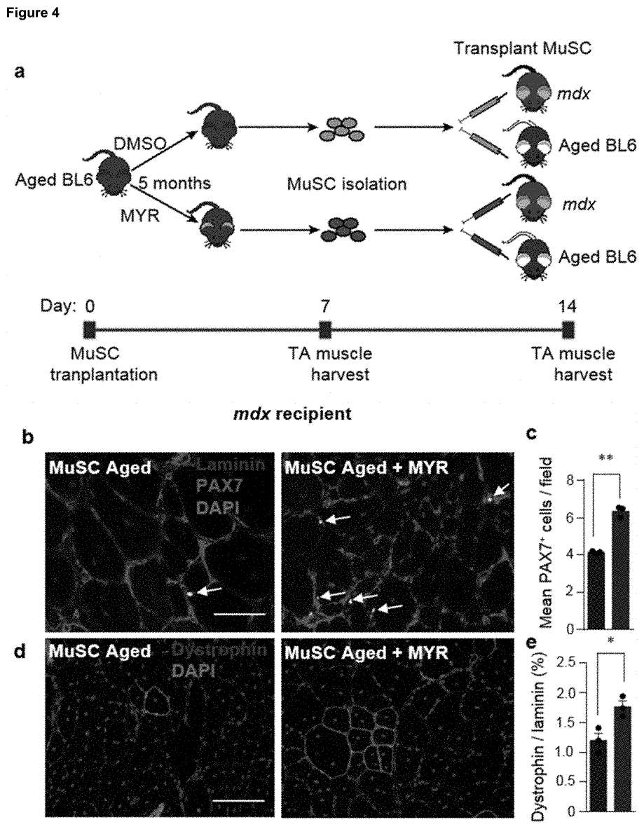

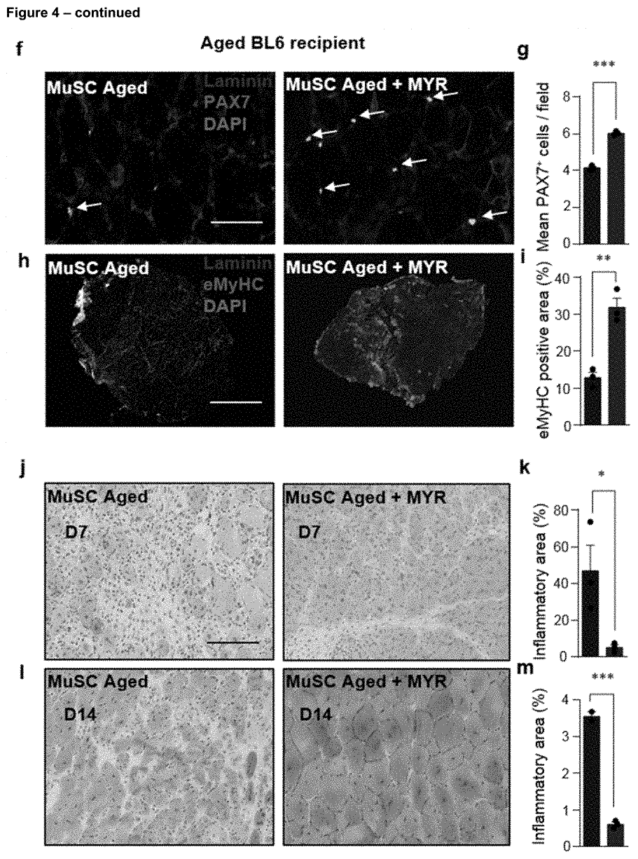

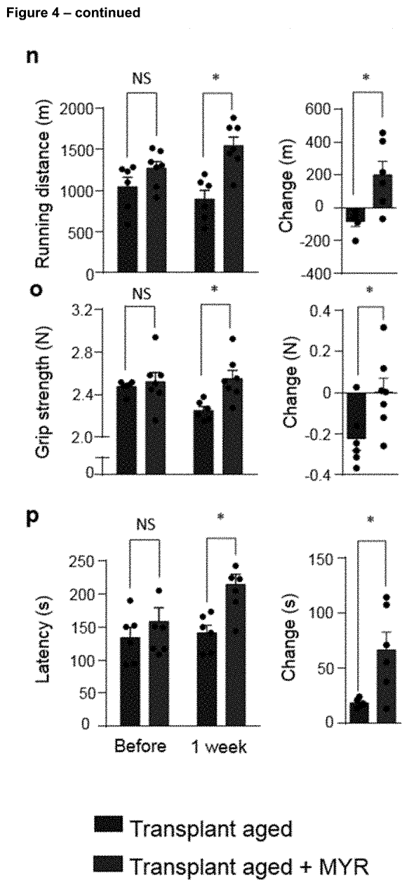

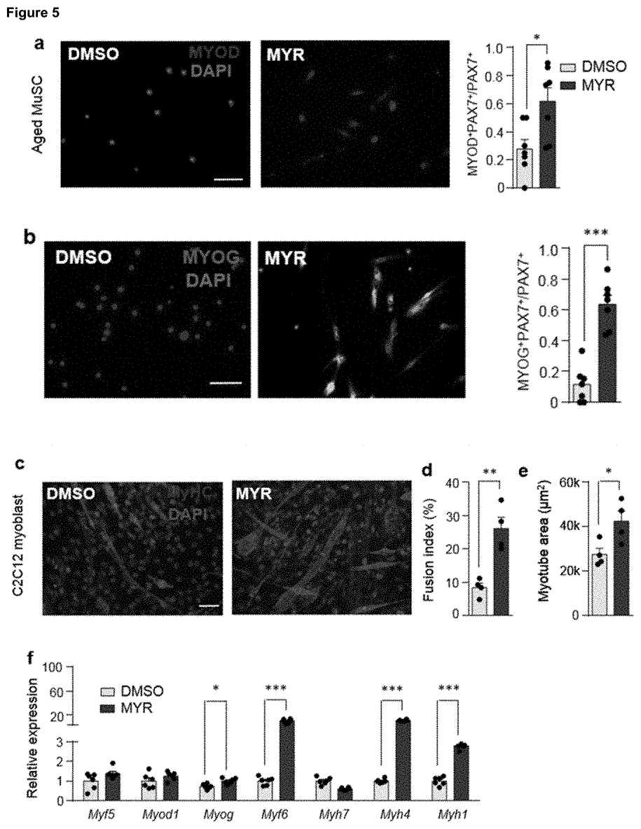

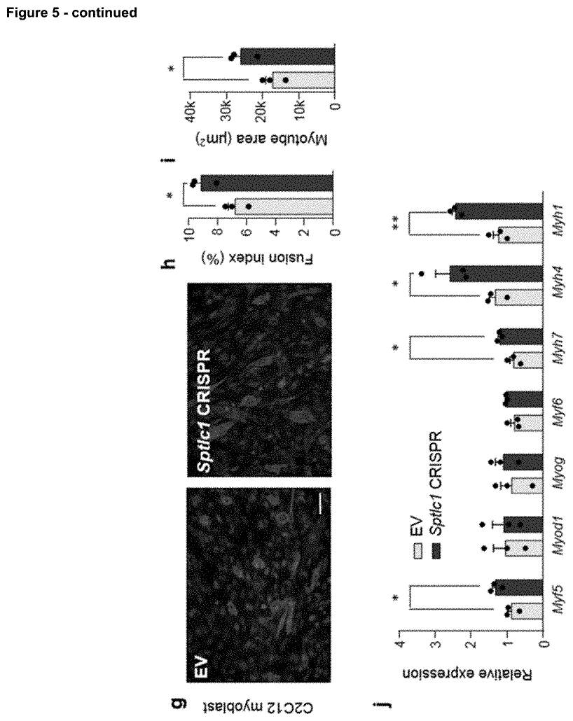

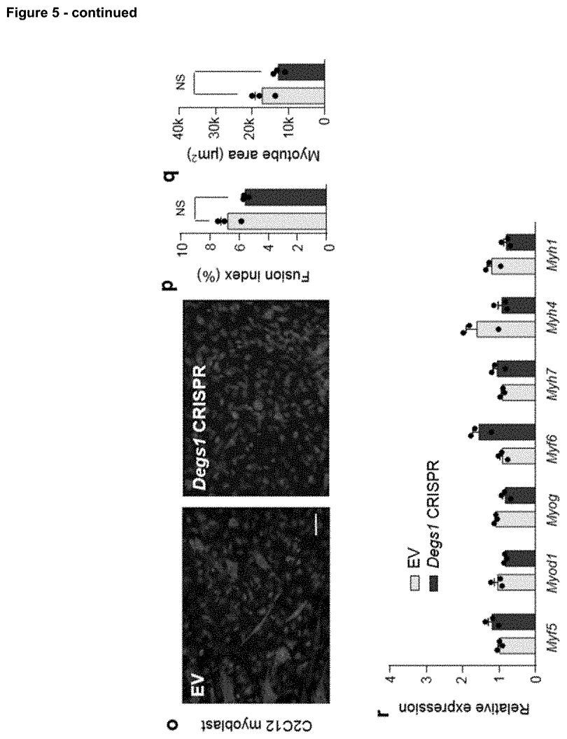

In mice, blocking sphingolipid de novo synthesis results in multiple benefits counteracting the effects of aging on MuSCs. Myriocin treatment increased the proliferative capacity of MuSCs, replenishing the age-depleted MuSC pool, and it appears to prime MuSCs to accelerated regeneration, as manifested by increased MYOD (myoblast determination protein 1) and MYOG (myogenin) protein levels (FIG. 5a-b) upon myriocin treatment, and even improvements in physiological test performance upon MuSC transplantation from myriocin treated mice (FIG. 4n-p). The increased myogenic potential is not limited to MuSCs, but extends to myoblasts as well, suggesting a broader myogenic effect of sphingolipid depletion on muscle progenitor cells.

Genetic analyses of the Helsinki Birth Cohort Study indicate that targeting sphingolipid de novo biosynthesis pathway has fitness-related benefits in humans. Individuals carrying the major alleles of SPTLC1 and SPTLC2 polymorphisms displayed both reduced mRNA expression in GTEx data, and improved age-related fitness in HBCS, represented by the SFT score. It appears that the SPTLC1 variant shows stronger associations with muscle strength, such as grip strength and arm curl, while the SPTLC2 variant shows stronger associations with flexibility, such as back scratch, and sit and reach. In summary, the overlap between the cis-eQTL and QTL signals hint that genetically decreased levels of SPTLC1 and SPTLC2 levels could lead to improved fitness which, consistent with the in vivo treatment of myriocin, indicates that pharmacological approaches to inactivate sphingolipid de novo synthesis in humans is a means for the treatment and prevention of aging related-diseases and aging. The findings show that the inhibition of the synthesis of sphingolipids is a therapeutic means due to improved fitness in aged myriocin treated mice, cell-autonomous effects on muscle differentiation, and evidence from human genetics.

In accordance with a preferred embodiment of the invention the aging related-disease is frailty, age-related multimorbidity or sarcopenia and preferably is frailty.

Frailty (or frailty syndrome) is a multidimensional geriatric syndrome that is characterised by cumulative decline in multiple body systems or functions, with pathogenesis involving physical as well as social dimensions. Frailty increases vulnerability to poor health outcomes such as disability, hospital admission, reduced quality of life and even death (Cruz-Jentoft el al (2019), Age Ageing; 48(1): 16-31). The frailty phenotype is defined as a distinct clinical syndrome meeting three or more of five phenotypic criteria: weakness, slowness, low level of physical activity, self-reported exhaustion, and unintentional weight loss (Chen et al. (2014), Clin Interv Aging. 2014; 9: 433-441). Also frailty is often associated with cognitive impairments. In particular, associations between frailty and cognitive decline and dementia have been shown. Furthermore, frailty is often associated with muscle atrophy (i.e. is the wasting or loss of muscle tissue) and/or sarcopenia.

Sarcopenia is a muscle disease (muscle failure) rooted in adverse muscle changes that accrue across a lifetime; sarcopenia is common among adults of older age but can also occur earlier in life (Cruz-Jentoft el al (2019), Age Ageing; 48(1): 16-31). Sarcopenia is a progressive and generalised skeletal muscle disorder that is associated with increased likelihood of adverse outcomes including falls, fractures, physical disability and mortality. The original operational definition of sarcopenia by EWGSOP (Writing Group for the European Working Group on Sarcopenia in Older People) was a major change at that time, as it added muscle function to former definitions based only on detection of low muscle mass. In its 2018 definition which also applies herein, EWGSOP2 uses low muscle strength as the primary parameter of sarcopenia. Muscle strength is presently the most reliable measure of muscle function. Specifically, sarcopenia is probable when low muscle strength is detected. A sarcopenia diagnosis is confirmed by the presence of low muscle quantity or quality. When low muscle strength, low muscle quantity/quality and low physical performance are all detected, sarcopenia is considered severe.

While the physical phenotype of frailty shows some overlap with sarcopenia (e.g. low grip strength, slow gait speed, and weight loss) frailty and sarcopenia are still distinct diseases (Cruz-Jentoft el al (2019), Age Ageing; 48(1): 16-31). The former is a geriatric syndrome while the later is a disease.

While sarcopenia is a contributor to the development of physical frailty, the syndrome of frailty represents a much broader concept. Frailty is seen as the decline over a lifetime in multiple physiological systems, resulting in negative consequences to physical, cognitive, and social dimensions (Cruz-Jentoft el al (2019), Age Ageing; 48(1): 16-31).

The age related-diseases also may be part of age-related multimorbidity, noting that multimorbidity is commonly defined as the presence of two or more chronic medical conditions in an individual. Multimorbidity can present several challenges in care particularly with higher numbers of coexisting conditions and related polypharmacy.

The examples herein below in particular demonstrate that inhibitors of sphingolipids are suitable for the treatment and prevention of frailty and sarcopenia. For this reason these two diseases are preferred, while frailty is most preferred.

In accordance with a more preferred embodiment of the invention the frailty is characterized by (i) sarcopenia and/or muscle atrophy, and (ii) cognitive impairment.

As discussed herein above, in subjects suffering from frailty often (i) sarcopenia and/or muscle atrophy, and (ii) cognitive impairment can be observed. The cognitive impairment is generally age-related cognitive impairment.

As also discussed, frailty is often characterized by weakness due to sarcopenia and/or muscle atrophy as well as memory loss due to cognitive impairment.

As can be taken from Example 9 it was surprisingly found that inhibitors of sphingolipids are particularly suitable for the treatment and prevention of frailty which is characterized by (i) sarcopenia and/or muscle atrophy, and (ii) cognitive impairment. This is because the treatment improves (i) sarcopenia and/or muscle atrophy, and (ii) cognitive impairment at the same time. In more detail, myriocin treated mice displayed reduced weakness, increased walking speed, reduced exhaustion, increased maximal oxygen consumption, higher body and muscle mass, and higher physical activity. All these parameters indicate an improvement of sarcopenia and/or muscle atrophy. Moreover, in an object recognition test, myriocin treated mice spent more time exploring both the familiar object and novel object as compared to their DMSO treated counterparts, yet proportionally more time exploring the novel objects than untreated mice. Thus, sphingolipid synthesis inhibition also improves memory.

In accordance with a more preferred embodiment of the invention the cognitive impairment is senile dementia.

Senile dementia is the mental loss of intellectual ability that is associated with old age. Senile dementia leads to a decrease in cognitive abilities, such as memory loss.

In accordance with a preferred embodiment of the invention the sphingolipids are sphinganines, sphingosines, ceramides, dihydroceramides, sphingomyelins, deoxysphingolipids (such as 1-deoxysphinganine) or any combination thereof.

Among this group ceramides are preferred since the examples herein below demonstrate a direct effect of the inhibition of the synthesis of ceramides.

Sphinganine, also termed dihydrosphingosine, is biosynthesized by a decarboxylating condensation of serine with palmitoyl-CoA to form a keto intermediate, which is then reduced by NADPH.

Sphingosine (2-amino-4-trans-octadecene-1,3-diol) is an 18-carbon amino alcohol with an unsaturated hydrocarbon chain, which forms a primary part of sphingolipids, a class of cell membrane lipids that include sphingomyelin, an important phospholipid.

Ceramides are a family of waxy lipid molecules. A ceramide is composed of sphingosine and a fatty acid.

A dihydroceramide can be converted into a ceramide by inserting the 4,5-trans-double bond to the sphingolipid backbone of dihydroceramide.

Sphingomyelin usually consists of phosphocholine and ceramide, or a phosphoethanolamine head group.

Deoxysphingolipids are formed by serine-palmitoyltransferase through the alternate use of alanine instead of its regular substrate serine.

In accordance with a further preferred embodiment of the invention the inhibitor is an inhibitor of one or more enzymes being involved in the biosynthesis of sphingolipids.

Sphingolipids are synthesized in a pathway that begins in the ER and is completed in the Golgi apparatus. The process of metabolism of sphingolipids has been studied extensively and most of the biochemical pathways of synthesis and degradation, including all the enzymes involved, have been determined successfully (Rao et al (2013), Journal of Lipids, Volume 2013, Article ID 178910).

In accordance with a more preferred embodiment of the invention the one or more enzymes is or are selected from SPTLC1, SPTLC2, SPTLC3, KDSR, CERS1, CERS2, CERS3, CERS4, CERS5, CERS6, DEGS1, SGMS1, SGMS2, SMPD1, SMPD2, SMPD3, SMPD4, ASAH1, ASAH2, ASAH2B, ASAH2C, ACER1, ACER2, ACER3, SPHK1 and SGPP1.

The full names of the enzymes will be provided herein below and the full names can also all be taken, for example, from the UniPort.org protein database. This database provides the scientific community with a freely accessible resource of protein sequence and functional information.

SPTLC1 to 3 are the serine palmitoyltransferase, long chain base subunits 1 to 3. Serine palmitoyltransferase, which consists of two different subunits, is the initial enzyme in sphingolipid biosynthesis. It converts L-serine and palmitoyl CoA to 3-oxosphinganine with pyridoxal 5′-phosphate as a cofactor.

KDSR is the 3-ketodihydrosphingosine reductase and catalyzes the reduction of 3-ketodihydrosphingosine (KDS) to dihydrosphingosine (DHS).

CERS1 to 6 are the ceramide synthases 1 to 6. CERS1 catalyzes the formation of ceramide from sphinganine and acyl-CoA substrates, with high selectivity toward stearoyl-CoA (octadecanoyl-CoA; C18:0-CoA). CERS2 Ceramide synthase that catalyzes formation of ceramide from sphinganine and acyl-CoA substrates, with high selectivity toward a very-long (C22:0-C24:0) chain as acyl donor.

CERS3 catalyzes the formation of ceramide from sphinganine and acyl-CoA substrates, also with high a selectivity toward very-long (C22:0-C24:0) and in addition ultra long chain (more than C26:0) as acyl donor. CERS4 catalyzes the formation of ceramide from sphinganine and acyl-CoA substrates, with high selectivity toward long and very-long chains (C18:0-C22:0) as acyl donor. CERS5 catalyzes the formation of ceramide from sphinganine and acyl-CoA substrates, with high selectivity toward palmitoyl-CoA (hexadecanoyl-CoA; C16:0-CoA) as acyl donor. Finally, CERS6 catalyzes the formation of ceramide from sphinganine and acyl-CoA substrates, with high selectivity toward palmitoyl-CoA (hexadecanoyl-CoA; C16:0-CoA) as acyl donor.

DEGS1 is the sphingolipid delta(4)-desaturase DES1 and has sphingolipid-delta-4-desaturase activity. It converts D-erythro-sphinganine to D-erythro-sphingosine (E-sphing-4-enine).

SGMS1 and 2 are phosphatidylcholine:ceramide cholinephosphotransferases 1 and 2. These sphingomyelin synthases synthesize the sphingolipid, sphingomyelin, through transfer of the phosphatidyl head group, phosphatidylcholine, on to the primary hydroxyl of ceramide.

SMPD1 to 4 are sphingomyelin phosphodiesterases 1 to 4 which convert sphingomyelin to ceramide.

ASAH1, 2, 2B, and 2C are ceramidases 1, 2, 2B and 2C that hydrolyze sphingolipid ceramides into sphingosine and free fatty acids at acidic or neutral pH.

ACER1 to 3 are ceramidases 1 to 3 that catalyze the hydrolysis of ceramides into sphingosine and free fatty acids at alkaline pH.

SPHK1 is sphingosine kinase 1 and phosphorylates sphingosine to sphingosine-1-phosphate (S1P).

SGPP1 is sphingosine-1-phosphate phosphatase 1 and specifically dephosphorylates sphingosine 1-phosphate (S1P), dihydro-S1P, and phyto-S1P.

In accordance with another more preferred embodiment of the invention the inhibitor inhibits (i) the expression of a nucleic acid molecule encoding one or more enzymes being involved in the biosynthesis of sphingolipids, or (ii) the enzymatic activity of an enzyme being involved in the biosynthesis of sphingolipids.

Expression (or gene expression) is the process by which information from a gene is used in the synthesis of a functional gene product, i.e. in the present case an enzyme being involved in the biosynthesis of sphingolipids. Expression involves the step of transcription of DNA into mRNA and the subsequent translation of the mRNA into protein. Hence, the inhibition of expression can be determined by determining the number of mRNA transcripts or protein in the presence and the absence of the inhibitor.

Enzyme activity can be expressed as the moles of substrate converted per unit time rate×reaction volume. Enzyme activity is a measure of the quantity of active enzyme present. The SI unit of enzyme activity is the katal, 1 katal=1 mol s⁻¹. Hence, the enzymatic activity can be tested in the presence and the absence of the inhibitor.

It is preferred with increasing preference that the inhibitor reduces the expression or enzymatic activity by at least 20%, at least 30%, at least 40%, at least 50%, at least 60%, at least 70%, at least 80% or at least 90%. Most preferably the expression or enzymatic activity is completely abolished.

It is preferred to inhibit the expression of a nucleic acid molecule encoding one or more enzymes being involved in the biosynthesis of sphingolipids by an inhibitor being capable of the introduction of a loss-of-function mutation into a nucleic acid molecule encoding one or more enzymes being involved in the biosynthesis of sphingolipids.

The efficiency of several inhibitors may be determined simultaneously in high-throughput formats. High-throughput assays, independently of being biochemical, cellular or other assays, generally may be performed in wells of microtiter plates, wherein each plate may contain 96, 384 or 1536 wells. Handling of the plates, including incubation at temperatures other than ambient temperature, and bringing into contact the test compounds with the assay mixture is preferably effected by one or more computer-controlled robotic systems including pipetting devices. In case large libraries of test compounds are to be screened and/or screening is to be effected within a short time, mixtures of, for example 10, 20, 30, 40, 50 or 100 test compounds may be added to each well. In case a well exhibits the expected activity, said mixture of test compounds may be de-convoluted to identify the one or more test compounds in said mixture giving rise to said activity.

In accordance with another more preferred embodiment of the invention (I) the inhibitor of (i) is selected from a small molecule, an aptamer, a siRNA, a shRNA, a miRNA, a ribozyme, an antisense nucleic acid molecule, a CRISPR-Cas9-based construct, a CRISPR-Cpf1-based construct, a meganuclease, a zinc finger nuclease, and a transcription activator-like (TAL) effector (TALE) nuclease, and/or (II) the inhibitor of (ii) is selected from a small molecule, an antibody or antibody mimetic, and an aptamer.

The “small molecule” as used herein is preferably an organic molecule. Organic molecules relate or belong to the class of chemical compounds having a carbon basis, the carbon atoms linked together by carbon-carbon bonds. The original definition of the term organic related to the source of chemical compounds, with organic compounds being those carbon-containing compounds obtained from plant or animal or microbial sources, whereas inorganic compounds were obtained from mineral sources. Organic compounds can be natural or synthetic. The organic molecule is preferably an aromatic molecule and more preferably a heteroaromatic molecule. In organic chemistry, the term aromaticity is used to describe a cyclic (ring-shaped), planar (flat) molecule with a ring of resonance bonds that exhibits more stability than other geometric or connective arrangements with the same set of atoms. Aromatic molecules are very stable, and do not break apart easily to react with other substances. In a heteroaromatic molecule at least one of the atoms in the aromatic ring is an atom other than carbon, e.g. N, S, or O. For all above-described organic molecules the molecular weight is preferably in the range of 200 Da to 1500 Da and more preferably in the range of 300 Da to 1000 Da.

Alternatively, the “small molecule” in accordance with the present invention may be an inorganic compound. Inorganic compounds are derived from mineral sources and include all compounds without carbon atoms (except carbon dioxide, carbon monoxide and carbonates). Preferably, the small molecule has a molecular weight of less than about 2000 Da, or less than about 1000 Da such as less than about 500 Da, and even more preferably less than about Da. The size of a small molecule can be determined by methods well-known in the art, e.g., mass spectrometry. The small molecules may be designed, for example, based on the crystal structure of the target molecule, where sites presumably responsible for the biological activity can be identified and verified in in vivo assays such as in vivo high-throughput screening (HTS) assays.

The term “antibody” as used in accordance with the present invention comprises, for example, polyclonal or monoclonal antibodies. Furthermore, also derivatives or fragments thereof, which still retain the binding specificity to the target are comprised in the term “antibody”. Antibody fragments or derivatives comprise, inter alia, Fab or Fab′ fragments, Fd, F(ab′)2, Fv or scFv fragments, single domain VH or V-like domains, such as VhH or V-NAR-domains, as well as multimeric formats such as minibodies, diabodies, tribodies or triplebodies, tetrabodies or chemically conjugated Fab′-multimers (see, for example, Harlow and Lane “Antibodies, A Laboratory Manual”, Cold Spring Harbor Laboratory Press, 198; Harlow and Lane “Using Antibodies: A Laboratory Manual” Cold Spring Harbor Laboratory Press, 1999; Altshuler E P, Serebryanaya D V, Katrukha A G. 2010, Biochemistry (Mosc)., vol. 75(13), 1584; Holliger P, Hudson P J. 2005, Nat Biotechnol., vol. 23(9), 1126). The multimeric formats in particular comprise bispecific antibodies that can simultaneously bind to two different types of antigen. Non-limiting examples of bispecific antibodies formats are Biclonics (bispecific, full length human IgG antibodies), DART (Dual-affinity Re-targeting Antibody) and BiTE (consisting of two single-chain variable fragments (scFvs) of different antibodies) molecules (Kontermann and Brinkmann (2015), Drug Discovery Today, 20(7):838-847).

The term “antibody” also includes embodiments such as chimeric (human constant domain, non-human variable domain), single chain and humanised (human antibody with the exception of non-human CDRs) antibodies.

Various techniques for the production of antibodies are well known in the art and described, e.g. in Harlow and Lane (see below) and Altshuler et al., 2010, loc. cit. Thus, polyclonal antibodies can be obtained from the blood of an animal following immunisation with an antigen in mixture with additives and adjuvants and monoclonal antibodies can be produced by any technique which provides antibodies produced by continuous cell line cultures. Examples for such techniques are described, e.g. in Harlow E and Lane D, Cold Spring Harbor Laboratory Press, 1988; Harlow E and Lane D, Using Antibodies: A Laboratory Manual, Cold Spring Harbor Laboratory Press, 1999 and include the hybridoma technique originally described by Kohler and Milstein, 1975, the trioma technique, the human B-cell hybridoma technique (see e.g. Kozbor D, 1983, Immunology Today, vol. 4, 7; Li J, et al. 2006, PNAS, vol. 103(10), 3557) and the EBV-hybridoma technique to produce human monoclonal antibodies (Cole et al., 1985, Alan R. Liss, Inc, 77-96). Furthermore, recombinant antibodies may be obtained from monoclonal antibodies or can be prepared de novo using various display methods such as phage, ribosomal, mRNA, or cell display. A suitable system for expression of the recombinant (humanised) antibodies may be selected from, for example, bacteria, yeast, insects, mammalian cell lines or transgenic animals or plants (see, e.g., U.S. Pat. No. 6,080,560; Holliger P, Hudson P J. 2005, Nat Biotechnol., vol. 23(9), 11265). Further, techniques described for the production of single chain antibodies (see, inter alia, U.S. Pat. No. 4,946,778) can be adapted to produce single chain antibodies specific for an epitope of a target. Surface plasmon resonance as employed in the BIAcore system can be used to increase the efficiency of phage antibodies.

As used herein, the term “antibody mimetics” refers to compounds which, like antibodies, can specifically bind antigens, such the herein-above described enzymes in the present case, but which are not structurally related to antibodies. Antibody mimetics are usually artificial peptides or proteins with a molar mass of about 3 to 20 kDa.

Aptamers are nucleic acid molecules or peptide molecules that bind a specific target molecule. Aptamers are usually created by selecting them from a large random sequence pool, but natural aptamers also exist in riboswitches. Aptamers can be used for both basic research and clinical purposes as macromolecular drugs. Aptamers can be combined with ribozymes to self-cleave in the presence of their target molecule. These compound molecules have additional research, industrial and clinical applications (Osborne et. al. (1997), Current Opinion in Chemical Biology, 1:5-9; Stull & Szoka (1995), Pharmaceutical Research, 12, 4:465-483).

Nucleic acid aptamers are nucleic acid species that normally consist of (usually short) strands of oligonucleotides. Typically, they have been engineered through repeated rounds of in vitro selection or equivalently, SELEX (systematic evolution of ligands by exponential enrichment) to bind to various molecular targets such as small molecules, proteins, nucleic acids, and even cells, tissues and organisms.

Peptide aptamers are usually peptides or proteins that are designed to interfere with other protein interactions inside cells. They consist of a variable peptide loop attached at both ends to a protein scaffold. This double structural constraint greatly increases the binding affinity of the peptide aptamer to levels comparable to an antibody's (nanomolar range). The variable peptide loop typically comprises 10 to 20 amino acids, and the scaffold may be any protein having good solubility properties. Currently, the bacterial protein Thioredoxin-A is the most commonly used scaffold protein, the variable peptide loop being inserted within the redox-active site, which is a -Cys-Gly-Pro-Cys-loop (SEQ ID NO: 55) in the wild protein, the two cysteins lateral chains being able to form a disulfide bridge. Peptide aptamer selection can be made using different systems, but the most widely used is currently the yeast two-hybrid system.

Aptamers offer the utility for biotechnological and therapeutic applications as they offer molecular recognition properties that rival those of the commonly used biomolecules, in particular antibodies. In addition to their discriminatory recognition, aptamers offer advantages over antibodies as they can be engineered completely in a test tube, are readily produced by chemical synthesis, possess desirable storage properties, and elicit little or no immunogenicity in therapeutic applications. Non-modified aptamers are cleared rapidly from the bloodstream, with a half-life of minutes to hours, mainly due to nuclease degradation and clearance from the body by the kidneys, a result of the aptamers' inherently low molecular weight. Unmodified aptamer applications currently focus on treating transient conditions such as blood clotting, or treating organs such as the eye where local delivery is possible. This rapid clearance can be an advantage in applications such as in vivo diagnostic imaging. Several modifications, such as 2′-fluorine-substituted pyrimidines, polyethylene glycol (PEG) linkage, fusion to albumin or other half life extending proteins etc. are available to scientists such that the half-life of aptamers can be increased for several days or even weeks.

In accordance with the present invention, the term “small interfering RNA (siRNA)”, also known as short interfering RNA or silencing RNA, refers to a class of 18 to 30, preferably 19 to 25, most preferred 21 to 23 or even more preferably 21 nucleotide-long double-stranded RNA molecules that play a variety of roles in biology. Most notably, siRNA is involved in the RNA interference (RNAi) pathway where the siRNA interferes with the expression of a specific gene. In addition to their role in the RNAi pathway, siRNAs also act in RNAi-related pathways, e.g. as an antiviral mechanism or in shaping the chromatin structure of a genome.

siRNAs naturally found in nature have a well defined structure: a short double-strand of RNA (dsRNA) with 2-nt 3′ overhangs on either end. Each strand has a 5′ phosphate group and a 3′ hydroxyl (—OH) group. This structure is the result of processing by dicer, an enzyme that converts either long dsRNAs or small hairpin RNAs into siRNAs. siRNAs can also be exogenously (artificially) introduced into cells to bring about the specific knockdown of a gene of interest. Essentially any gene for which the sequence is known can thus be targeted based on sequence complementarity with an appropriately tailored siRNA. The double-stranded RNA molecule or a metabolic processing product thereof is capable of mediating target-specific nucleic acid modifications, particularly RNA interference and/or DNA methylation. Exogenously introduced siRNAs may be devoid of overhangs at their 3′ and 5′ ends, however, it is preferred that at least one RNA strand has a 5′- and/or 3′-overhang. Preferably, one end of the double-strand has a 3′-overhang from 1 to 5 nucleotides, more preferably from 1 to 3 nucleotides and most preferably 2 nucleotides. The other end may be blunt-ended or has up to 6 nucleotides 3′-overhang. In general, any RNA molecule suitable to act as siRNA is envisioned in the present invention. The sequence of the 2-nt 3′ overhang makes a small contribution to the specificity of target recognition restricted to the unpaired nucleotide adjacent to the first base pair (Elbashir et al. 2001). 2′-deoxynucleotides in the 3′ overhangs are as efficient as ribonucleotides, but are often cheaper to synthesize and probably more nuclease resistant. Delivery of siRNA may be accomplished using any of the methods known in the art, for example by combining the siRNA with saline and administering the combination intravenously or intranasally or by formulating siRNA in glucose (such as for example 5% glucose) or cationic lipids and polymers can be used for siRNA delivery in vivo through systemic routes either intravenously (IV) or intraperitoneally (IP) (Fougerolles et al. (2008), Current Opinion in Pharmacology, 8:280-285; Lu et al. (2008), Methods in Molecular Biology, vol. 437: Drug Delivery Systems—Chapter 3: Delivering Small Interfering RNA for Novel Therapeutics). Nowadays, siRNAs are typically heavily modified in order to increase their lifetime.

A short hairpin RNA (shRNA) is a sequence of RNA that makes a tight hairpin turn that can be used to silence gene expression via RNA interference. shRNA uses a vector introduced into cells and utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs which match the siRNA that is bound to it. si/shRNAs to be used in the present invention are preferably chemically synthesized using appropriately protected ribonucleoside phosphoramidites and a conventional DNA/RNA synthesizer. Suppliers of RNA synthesis reagents are Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK). Most conveniently, siRNAs or shRNAs are obtained from commercial RNA oligo synthesis suppliers, which sell RNA-synthesis products of different quality and costs. In general, the RNAs applicable in the present invention are conventionally synthesized and are readily provided in a quality suitable for RNAi.

Further molecules effecting RNAi include, for example, microRNAs (miRNA). Said RNA species are single-stranded RNA molecules. Endogenously present miRNA molecules regulate gene expression by binding to a complementary mRNA transcript and triggering of the degradation of said mRNA transcript through a process similar to RNA interference. Accordingly, exogenous miRNA may be employed as an inhibitor of an enzyme after introduction into the respective cells.

A ribozyme (from ribonucleic acid enzyme, also called RNA enzyme or catalytic RNA) is an RNA molecule that catalyses a chemical reaction. Many natural ribozymes catalyse either their own cleavage or the cleavage of other RNAs, but they have also been found to catalyse the aminotransferase activity of the ribosome. Non-limiting examples of well-characterised small self-cleaving RNAs are the hammerhead, hairpin, hepatitis delta virus, and in vitro-selected lead-dependent ribozymes, whereas the group I intron is an example for larger ribozymes. The principle of catalytic self-cleavage has become well established in recent years. The hammerhead ribozymes are characterised best among the RNA molecules with ribozyme activity. Since it was shown that hammerhead structures can be integrated into heterologous RNA sequences and that ribozyme activity can thereby be transferred to these molecules, it appears that catalytic antisense sequences for almost any target sequence can be created, provided the target sequence contains a potential matching cleavage site. The basic principle of constructing hammerhead ribozymes is as follows: A region of interest of the RNA, which contains the GUC (or CUC) triplet, is selected. Two oligonucleotide strands, each usually with 6 to 8 nucleotides, are taken and the catalytic hammerhead sequence is inserted between them. The best results are usually obtained with short ribozymes and target sequences.

A recent development, also useful in accordance with the present invention, is the combination of an aptamer, recognizing a small compound, with a hammerhead ribozyme. The conformational change induced in the aptamer upon binding the target molecule can regulate the catalytic function of the ribozyme.

The term “antisense nucleic acid molecule”, as used herein, refers to a nucleic acid which is complementary to a target nucleic acid. An antisense molecule in accordance with the invention is capable of interacting with the target nucleic acid, more specifically it is capable of hybridizing with the target nucleic acid. Due to the formation of the hybrid, transcription of the target gene(s) and/or translation of the target mRNA is reduced or blocked. Standard methods relating to antisense technology have been described (see, e.g., Melani et al., Cancer Res. (1991) 51:2897-2901).

CRISPR/Cas9, as well as CRISPR-Cpf1, technologies are applicable in nearly all cells/model organisms and can be used for knock out mutations, chromosomal deletions, editing of DNA sequences and regulation of gene expression. The regulation of the gene expression can be manipulated by the use of a catalytically dead Cas9 enzyme (dCas9) that is conjugated with a transcriptional repressor to repress transcription a specific target gene. Similarly, catalytically inactive, “dead” Cpf1 nuclease (CRISPR from Prevotella and Francisella-1) can be fused to synthetic transcriptional repressors or activators to downregulate endogenous promoters, e.g. the promoter which controls the expression of a target gene. Alternatively, the DNA-binding domain of zinc finger nucleases (ZFNs) or transcription activator-like effector nucleases (TALENs) can be designed to specifically recognize the target gene or its promoter region or its 5′-UTR thereby inhibiting the expression of the target gene.

In accordance with an even more preferred embodiment of the invention the antibody mimetic is selected from affibodies, adnectins, anticalins, DARPins, avimers, nanofitins, affilins, Kunitz domain peptides, Fynomers®, trispecific binding molecules and probodies.

The term “affibody”, as used herein, refers to a family of antibody mimetics which is derived from the Z-domain of staphylococcal protein A. Structurally, affibody molecules are based on a three-helix bundle domain which can also be incorporated into fusion proteins. In itself, an affibody has a molecular mass of around 6 kDa and is stable at high temperatures and under acidic or alkaline conditions. Target specificity is obtained by randomisation of 13 amino acids located in two alpha-helices involved in the binding activity of the parent protein domain (Feldwisch J, Tolmachev V.; (2012) Methods Mol Biol. 899:103-26).

The term “adnectin” (also referred to as “monobody”), as used herein, relates to a molecule based on the 10th extracellular domain of human fibronectin III (10Fn3), which adopts an Ig-like β-sandwich fold of 94 residues with 2 to 3 exposed loops, but lacks the central disulphide bridge (Gebauer and Skerra (2009) Curr Opinion in Chemical Biology 13:245-255). Adnectins with the desired target specificity can be genetically engineered by introducing modifications in specific loops of the protein.

The term “anticalin”, as used herein, refers to an engineered protein derived from a lipocalin (Beste G, Schmidt F S, Stibora T, Skerra A. (1999) Proc Natl Acad Sci USA. 96(5):1898-903; Gebauer and Skerra (2009) Curr Opinion in Chemical Biology 13:245-255). Anticalins possess an eight-stranded β-barrel which forms a highly conserved core unit among the lipocalins and naturally forms binding sites for ligands by means of four structurally variable loops at the open end. Anticalins, although not homologous to the IgG superfamily, show features that so far have been considered typical for the binding sites of antibodies: (i) high structural plasticity as a consequence of sequence variation and (ii) elevated conformational flexibility, allowing induced fit to targets with differing shape.

As used herein, the term “DARPin” refers to a designed ankyrin repeat domain (166 residues), which provides a rigid interface arising from typically three repeated β-turns. DARPins usually carry three repeats corresponding to an artificial consensus sequence, wherein six positions per repeat are randomised. Consequently, DARPins lack structural flexibility (Gebauer and Skerra, 2009).

The term “avimer”, as used herein, refers to a class of antibody mimetics which consist of two or more peptide sequences of 30 to 35 amino acids each, which are derived from A-domains of various membrane receptors and which are connected by linker peptides. Binding of target molecules occurs via the A-domain and domains with the desired binding specificity can be selected, for example, by phage display techniques. The binding specificity of the different A-domains contained in an avimer may, but does not have to be identical (Weidle U H, et al., (2013), Cancer Genomics Proteomics; 10(4):155-68).

A “nanofitin” (also known as affitin) is an antibody mimetic protein that is derived from the DNA binding protein Sac7d of Sulfolobus acidocaldarius. Nanofitins usually have a molecular weight of around 7 kDa and are designed to specifically bind a target molecule by randomising the amino acids on the binding surface (Mouratou B, Behar G, Paillard-Laurance L, Colinet S, Pecorari F., (2012) Methods Mol Biol.; 805:315-31).

The term “affilin”, as used herein, refers to antibody mimetics that are developed by using either gamma-B crystalline or ubiquitin as a scaffold and modifying amino-acids on the surface of these proteins by random mutagenesis. Selection of affilins with the desired target specificity is effected, for example, by phage display or ribosome display techniques. Depending on the scaffold, affilins have a molecular weight of approximately 10 or 20 kDa. As used herein, the term affilin also refers to di- or multimerised forms of affilins (Weidle U H, et al., (2013), Cancer Genomics Proteomics; 10(4):155-68).

A “Kunitz domain peptide” is derived from the Kunitz domain of a Kunitz-type protease inhibitor such as bovine pancreatic trypsin inhibitor (BPTI), amyloid precursor protein (APP) or tissue factor pathway inhibitor (TFPI). Kunitz domains have a molecular weight of approximately 6 kDA and domains with the required target specificity can be selected by display techniques such as phage display (Weidle et al., (2013), Cancer Genomics Proteomics; 10(4):155-68).

As used herein, the term “Fynomer®” refers to a non-immunoglobulin-derived binding polypeptide derived from the human Fyn SH3 domain. Fyn SH3-derived polypeptides are well-known in the art and have been described e.g. in Grabulovski et al. (2007) JBC, 282, p. 3196-3204, WO 2008/022759, Bertschinger et al (2007) Protein Eng Des Sel 20(2):57-68, Gebauer and Skerra (2009) Curr Opinion in Chemical Biology 13:245-255, or Schlatter et al. (2012), MAbs 4:4, 1-12).

The term “trispecific binding molecule” as used herein refers to a polypeptide molecule that possesses three binding domains and is thus capable of binding, preferably specifically binding to three different epitopes. At least one of these three epitopes is an epitope of the protein of the fourth aspect of the invention. The two other epitopes may also be epitopes of the protein of the fourth aspect of the invention or may be epitopes of one or two different antigens. The trispecific binding molecule is preferably a TriTac. A TriTac is a T-cell engager for solid tumors which is comprised of three binding domains being designed to have an extended serum half-life and be about one-third the size of a monoclonal antibody.

As used herein, the term “probody” refers to a protease-activatable antibody prodrug. A probody consists of an authentic IgG heavy chain and a modified light chain. A masking peptide is fused to the light chain through a peptide linker that is cleavable by tumor-specific proteases. The masking peptide prevents the probody binding to healthy tissues, thereby minimizing toxic side effects.

In accordance with a further more preferred embodiment of the invention the small molecule of (ii) ismyriocin or a small molecule inhibitor selected from Table A, or a salt or ester of any of these inhibitors.

Myriocin (2-Amino-3,4-dihydroxy-2-(hydroxymethyl)-14-oxoicos-6-enoic acid), also known as antibiotic ISP-1 and thermozymocidin, is an atypical amino acid and an antibiotic derived from certain thermophilic fungi. Myriocin is a very potent inhibitor of serine palmitoyltransferase, the first step in sphingosine biosynthesis. Myriocin is preferably administered intraperitoneally, since this avoids potential side effects by oral administration. Myriocin or a salt or ester thereof is the most preferred small molecule inhibitor since it is used in the examples.

However, next to myriocin several other small molecule inhibitors of enzymes being involved in the synthesis of sphingolipids are known. Non-limiting but preferred examples are shown in Table A.

TABLE A
Small molecule inhibitors of enzymes being involved in the synthesis of sphingolipids

| Compound | Reference |
|---|---|
| 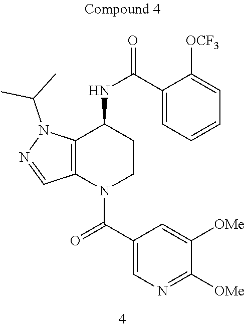 [SMILES: COc1cc(C(=O)N2CC[C@H](NC(=O)c3ccccc3OC(F)(F)F)c3c2cnn3C(C)C)cnc1OC] | Bioorg Med Chem. 2018 May 15;26(9):2452-2465. doi: 10.1016/j.bmc.2018.04.008. Epub 2018 Apr. 4. Discovery of novel serine palmitoyltransferase inhibitors as cancer therapeutic agents. Kojima T |
| 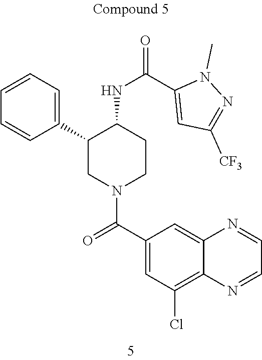 [SMILES: Cn1nc(C(F)(F)F)cc1C(=O)N[C@@H]1CCN(C(=O)c2cc(Cl)c3nccnc3c2)C[C@@H]1c1ccccc1] | Bioorg Med Chem. 2018 May 15;26(9):2452-2465. doi: 10.1016/j.bmc.2018.04.008. Epub 2018 Apr. 4. Discovery of novel serine palmitoyltransferase inhibitors as cancer therapeutic agents. Kojima T |
| 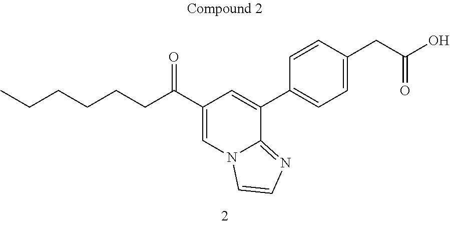 [SMILES: CCCCCCC(=O)c1cc(-c2ccc(CC(=O)O)cc2)c2nccn2c1] | Bioorg Med Chem. 2018 May 15;26(9):2452-2465. doi: 10.1016/j.bmc.2018.04.008. Epub 2018 Apr. 4. Discovery of novel serine palmitoyltransferase inhibitors as cancer therapeutic agents. Kojima T |
| 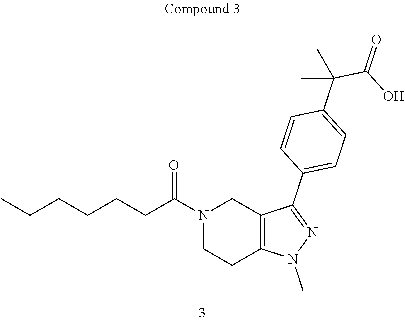 [SMILES: CCCCCCC(=O)N1CCc2c(c(-c3ccc(C(C)(C)C(=O)O)cc3)nn2C)C1] | Bioorg Med Chem. 2018 May 15;26(9):2452-2465. doi: 10.1016/j.bmc.2018.04.008. Epub 2018 Apr. 4. Discovery of novel serine palmitoyltransferase inhibitors as cancer therapeutic agents. Kojima T |
| 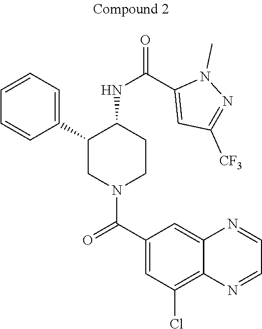 [SMILES: Cn1nc(C(F)(F)F)cc1C(=O)N[C@@H]1CCN(C(=O)c2cc(Cl)c3nccnc3c2)C[C@@H]1c1ccccc1] | Biochem Biophys Res Common. 2017 Mar. 11;484(3):493- 500. doi: 10.1016/j.bbrc.2017.01.075. Epub 2017 Jan. 17. Antitumor activity of a novel and orally available inhibitor of serine palmitoyltransferase. Yaguchi |
| L-serine, D-serine, D-threonine, O-methyl- | WO 20111/04298A1 |
| D, L-serine, sphingofungin B, myriocin, | |
| lipoxamycin, viridiofungin A, cycloserine, D- | |
| alanine and β-chloroalanine | |
| Mycestericins (e.g. mycestericins A-G) | Sasaki, S. et al., J. Antibiot. 47: 420-33 (1994) |
| Sphingofungins (e.g. the sphingofungins A- | VanMiddlesworth F., et al., J. Antibiotics 45: 861-7 (1992) |
| F) | |
| Cycloserine, D-serine, viridiofungin A and | — |
| lipoxamycin | |
| Alverine | (Functional inhibitors of acid sphingomyelinase) FIASMAS |
| Amiodarone | FIAMAS |
| Amitriptyline | FIAMAS |
| Amlodipine | FIAMAS |
| Aprindine | FIAMAS |
| Astemizole | FIAMAS |
| AY-9944 | FIAMAS |
| Benzatropine | FIAMAS |
| Bepridil | FIAMAS |
| Biperiden | FIAMAS |
| Camylofin | FIAMAS |
| Carvedilol | FIAMAS |
| Cepharanthine | FIAMAS |
| Chlorpromazine | FIAMAS |
| Chlorprothixene | FIAMAS |
| Cinnarizine | FIAMAS |
| Clemastine | FIAMAS |
| Clofazimine | FIAMAS |
| Clomiphene | FIAMAS |
| Clomipramine | FIAMAS |
| Cloperastine | FIAMAS |
| Conessine | FIAMAS |
| Cyclobenzaprine | FIAMAS |
| Cyproheptadine | FIAMAS |
| Desipramine | FIAMAS |
| Desloratadine | FIAMAS |
| Dicycloverine | FIAMAS |
| Dicyclomine | FIAMAS |
| Dilazep | FIAMAS |
| Dimebon | FIAMAS |
| Doxepine | FIAMAS |
| Drofenine | FIAMAS |
| Emetine | FIAMAS |
| Fendiline | FIAMAS |
| Flunarizine | FIAMAS |
| Fluoxetine | FIAMAS |
| Flupentixol | FIAMAS |
| Fluphenazine | FIAMAS |
| Fluvoxamine | FIAMAS |
| Hydroxyzine | FIAMAS |
| Imipramine | FIAMAS |
| Lofepramine | FIAMAS |
| Loperamid | FIAMAS |
| Loratadin | FIAMAS |
| Maprotiline | FIAMAS |
| Mebeverine | FIAMAS |
| Mebhydrolin | FIAMAS |
| Mepacrine | FIAMAS |
| Mibefradil | FIAMAS |
| Norfluoxetine | FIAMAS |
| Nortriptyline | FIAMAS |
| Paroxetine | FIAMAS |
| Penfluridol | FIAMAS |
| Perhexiline | FIAMAS |
| Perphanazine | FIAMAS |
| Pimethixene | FIAMAS |
| Pimozide | FIAMAS |
| Profenamine | FIAMAS |
| Promazine | FIAMAS |
| Promethazine | FIAMAS |
| Protriptyline | FIAMAS |
| Sertindole | FIAMAS |
| Sertraline | FIAMAS |
| Solasodine | FIAMAS |
| Suloctidil | FIAMAS |
| Tamoxifen | FIAMAS |
| Terfenadine | FIAMAS |
| Thioridazine | FIAMAS |
| Tomatidine | FIAMAS |
| Trifluoperazin | FIAMAS |
| Triflupromazine | FIAMAS |
| Trimipramine | FIAMAS |
| Zolantidine | FIAMAS |
| P053 | Turner, 2018, Nat Comm 9, Turner, 2018, Art No. 3165 |
| ST1058 | Biochimie, Volume 94, Issue 2, February 2012, Pages 558- |
| | 565. Inhibitors of specific ceramide synthases. Schiffmann. |
| ST1074 | Biochimie, Volume 94, Issue 2, February 2012, Pages 558- |
| | 565. Inhibitors of specific ceramide synthases. Schiffmann. |
| ST1072 | Biochimie, Volume 94, Issue 2, February 2012, Pages 558- |
| | 565. Inhibitors of specific ceramide synthases. Schiffmann. |
| ST1060 | Biochimie, Volume 94, Issue 2, February 2012, Pages 558-565. |
| | Inhibitors of specific ceramide synthases. Schiffmann. |
| Fumonisin B | Fumsine B1: (2S,2′S)-2,2′- |
| | {[(5S,6R,7R,9R,11S,16R,18S,19S)-19-Amino-11,16,18- |
| | trihydroxy-5,9-dimethylicosane-6,7-diyl]bis[oxy(2-oxoethane- |
| | 2,1-diyl)]}disuccinic acid |
| Ceranib-2 | |
| Ceranib 1 | |
| Oleylethanolamide | |
| Ceramidastin | |
| LCL521 | |
| LCL351 | INTERNATIONAL JOURNAL OF MOLECULAR MEDICINE |
| | 41: 2450-2460, 2018. Sphingosine kinase inhibitors: A patent |
| | review. Mengda Cao |
| SLR080811 | INTERNATIONAL JOURNAL OF MOLECULAR MEDICINE |
| | 41: 2450-2460, 2018. Sphingosine kinase inhibitors: A patent |
| | review. Mengda Cao |
| SLP120701 | INTERNATIONAL JOURNAL OF MOLECULAR MEDICINE |
| | 41: 2450-2460, 2018. Sphingosine kinase inhibitors: A patent review. |
| | Mengda Cao |
| 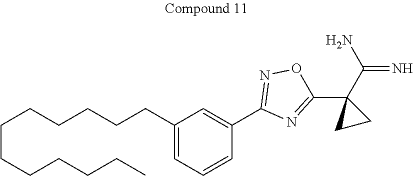 | INTERNATIONAL JOURNAL OF MOLECULAR MEDICINE 41: 2450-2460, 2018. Sphingosine kinase inhibitors: A patent review. Mengda Cao |
| VPC96091 | INTERNATIONAL JOURNAL OF MOLECULAR MEDICINE |
| | 41: 2450-2460, 2018. Sphingosine kinase inhibitors: A patent review. |
| | Mengda Cao |
| VPC96077 | INTERNATIONAL JOURNAL OF MOLECULAR MEDICINE |
| | 41: 2450-2460, 2018. Sphingosine kinase inhibitors: A patent review. |
| | Mengda Cao |
| 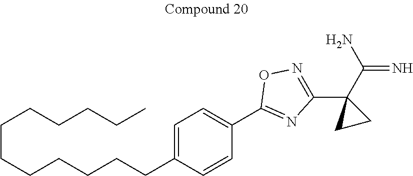 | INTERNATIONAL JOURNAL OF MOLECULAR MEDICINE 41: 2450-2460, 2018. Sphingosine kinase inhibitors: A patent review. Mengda Cao |
| 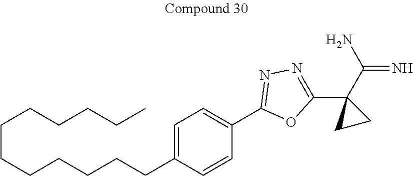 | INTERNATIONAL JOURNAL OF MOLECULAR MEDICINE 41: 2450-2460, 2018. Sphingosine kinase inhibitors: A patent review. Mengda Cao |
| SLC5091592 | INTERNATIONAL JOURNAL OF MOLECULAR MEDICINE |
| | 41: 2450-2460, 2018. Sphingosine kinase inhibitors: A patent |
| | review. Mengda Cao |
| SLC5081308 | INTERNATIONAL JOURNAL OF MOLECULAR MEDICINE |
| | 41: 2450-2460, 2018. Sphingosine kinase inhibitors: A patent |
| | review. Mengda Cao |
| SLC511312 | INTERNATIONAL JOURNAL OF MOLECULAR MEDICINE |
| | 41: 2450-2460, 2018. Sphingosine kinase inhibitors: A patent |
| | review. Mengda Cao |
| FTY720 | INTERNATIONAL JOURNAL OF MOLECULAR MEDICINE |
| | 41: 2450-2460, 2018. Sphingosine kinase inhibitors: A patent |
| | review. Mengda Cao |
| FTY720-OCH3 | INTERNATIONAL JOURNAL OF MOLECULAR MEDICINE |
| | 41: 2450-2460, 2018. Sphingosine kinase inhibitors: A patent |
| | review. Mengda Cao |
| SKI-I | INTERNATIONAL JOURNAL OF MOLECULAR MEDICINE |
| | 41: 2450-2460, 2018. Sphingosine kinase inhibitors: A patent |
| | review. Mengda Cao |
| SKI-II | INTERNATIONAL JOURNAL OF MOLECULAR MEDICINE |
| | 41: 2450-2460, 2018. Sphingosine kinase inhibitors: A patent |
| | review. Mengda Cao |
| SKI-III | INTERNATIONAL JOURNAL OF MOLECULAR MEDICINE |
| | 41: 2450-2460, 2018. Sphingosine kinase inhibitors: A patent |
| | review. Mengda Cao |
| SKI-IV | INTERNATIONAL JOURNAL OF MOLECULAR MEDICINE |
| | 41: 2450-2460, 2018. Sphingosine kinase inhibitors: A patent |
| | review. Mengda Cao |
| CNT2130 | Centaurus therapeutics, Discovery Park, Kent, UK- |
| | compound 2130. |

In accordance with another more preferred embodiment of the invention the inhibitor edits the genome at a location encoding an enzyme being involved in the biosynthesis of sphingolipids.

Genome editing (or genome engineering, or gene editing) is a type of genetic engineering in which DNA is inserted, deleted, modified or replaced in the genome of a living organism. In accordance with the invention the genome editing results in the inhibition of the expression of one or more enzymes being involved in the biosynthesis of sphingolipids.

Inhibitors provided as inhibiting nucleic acid molecules that target the gene encoding the enzyme of interest or a regulatory molecule involved in its expression and result in genome editing are therefore also envisaged herein. Such inhibitors, which reduce or abolish the expression of the target gene or a regulatory molecule include, without being limiting, meganucleases, zinc finger nucleases and transcription activator-like (TAL) effector (TALE) nucleases. Such methods are described in Silva et al., Curr Gene Ther. 2011; 11(1):11-27; Miller et al., Nature biotechnology. 2011; 29(2):143-148, and Klug, Annual review of biochemistry. 2010; 79:213-231.

In accordance with another preferred embodiment of the invention the aging-related disease is Alzheimer's disease, dementia, Parkinson's disease, Huntington's disease, or amyotrophic lateral sclerosis.

Alzheimer's disease is an irreversible, progressive brain disorder that slowly destroys memory and thinking skills, and, eventually, the ability to carry out the simplest tasks. In most people with Alzheimer's, symptoms first appear in their mid-60s.

Dementia is a category of brain diseases that cause a long-term and often gradual decrease in the ability to think and remember that is severe enough to affect a person's daily functioning. Other common symptoms include emotional problems, difficulties with language, and a decrease in motivation. The dementia is preferably senile dementia as described herein above.

Parkinson's disease (PD) is a long-term degenerative disorder of the central nervous system that mainly affects the motor system. As the disease worsens, non-motor symptoms become more common. The symptoms usually emerge slowly.

Huntington's disease is an inherited disorder that results in the death of brain cells. The earliest symptoms are often subtle problems with mood or mental abilities. A general lack of coordination and an unsteady gait often follow. As the disease advances, uncoordinated, jerky body movements become more apparent. Physical abilities gradually worsen until coordinated movement becomes difficult and the person is unable to talk. Mental abilities generally decline. Symptoms usually begin between 30 and 50 years of age.

Amyotrophic lateral sclerosis (ALS) (also known as motor neurone disease (MND) or Lou Gehrig's disease) is a specific disease that causes the death of neurons controlling voluntary muscles. ALS is characterized by stiff muscles, muscle twitching, and gradually worsening weakness due to muscles decreasing in size. ALS may begin with weakness in the arms or legs, or with difficulty speaking or swallowing. About half of the people affected develop at least mild difficulties with thinking and behaviour and most people experience pain. Most eventually lose the ability to walk, use their hands, speak, swallow, and breathe. The peak age of onset is 58 to 63 years for sporadic ALS and 47 to 52 years for familiar ALS.

In accordance with a further preferred embodiment of the invention the aging-related disease is a muscle disease.

Muscle diseases are a category of diseases and disorders that affect the muscle system. Diseases and disorders that result from direct abnormalities of the muscles are called primary muscle diseases; those that can be traced as symptoms or manifestations of disorders of nerves or other systems are not properly classified as primary muscle diseases. The muscle diseases are therefore preferably primary muscle diseases. Muscle diseases can cause weakness, pain or even paralysis.

In accordance with a more preferred embodiment of the invention the muscle disease is inclusion body myositis or a muscular dystrophy, preferably Duchenne muscular dystrophy.

Inclusion body myositis (IBM) (sometimes called sporadic inclusion body myositis, sIBM) is the most common inflammatory muscle disease in older adults. The disease is characterized by slowly progressive weakness and wasting of both distal and proximal muscles, most apparent in the finger flexors and knee extensors.

Muscular dystrophy (MD) is a group of muscle diseases that results in increasing weakening and breakdown of skeletal muscles over time. The disorders differ in which muscles are primarily affected, the degree of weakness, how fast they worsen, and when symptoms begin. Many people will eventually become unable to walk. Duchenne muscular dystrophy (DMD) is a severe type of muscular dystrophy. The symptom of muscle weakness usually begins around the age of four in boys and worsens quickly. Typically muscle loss occurs first in the thighs and pelvis followed by those of the arms.

In accordance with a yet further preferred embodiment of the invention the aging-related disease is a proteostatic diseases.

Proteostasis or protein homeostasis is a cellular network essential for the strict control of protein synthesis, protein folding, maintenance of conformation, and protein degradation. Depending on the proteomic demands, the expression of the proteostasis network (PN) differs in cells and tissues.

A proteostatic diseases is characterized by impaired or even a loss of proteostasis. Impaired proteostasis leads to protein aggregation that is responsible for dysregulation of the chaperone and co-chaperone levels.

Defects or alteration in the proteostasis pathways are linked to a number of diseases. Non-limiting bit preferred diseases are neurodegenerative diseases (e.g. Alzheimer disease or amyotrophic lateral sclerosis (ALS)), cardiac diseases, cognitive impairment, hereditary skeletal disorders (e.g. mutations in the mutations in the type 1 procollagen genes), dystrophies and rhodopsin retinitis pigmentosa (Labbadia and Morimoto (2015), Annu Rev Biochem., 84:435-46).

As regards the embodiments characterized in this specification, in particular in the claims, it is intended that each embodiment mentioned in a dependent claim is combined with each embodiment of each claim (independent or dependent) said dependent claim depends from. For example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

The figures show.

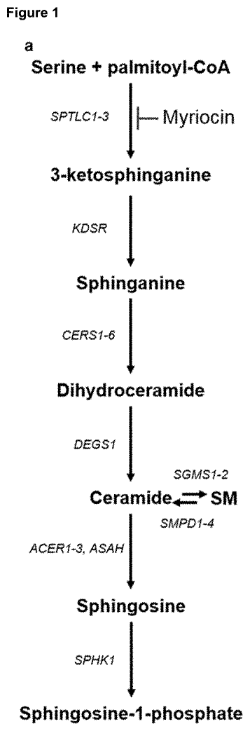

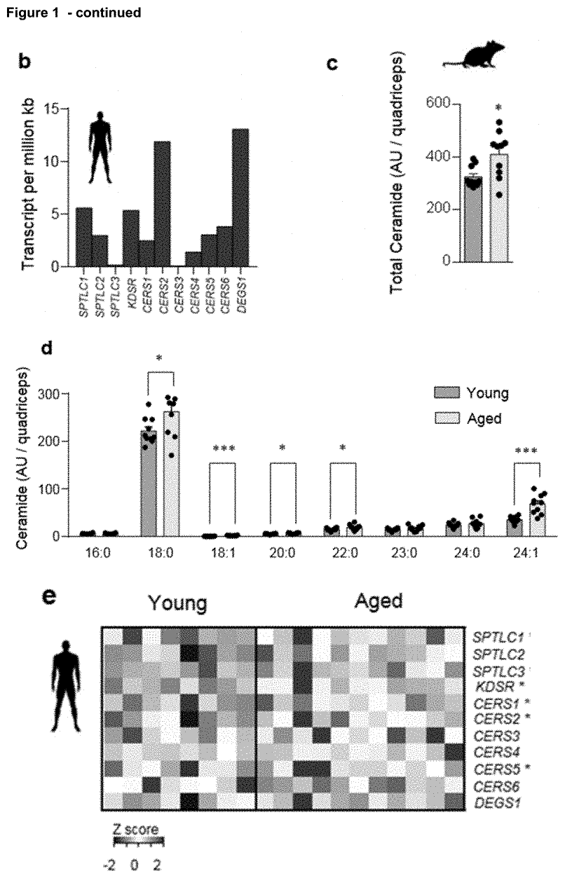

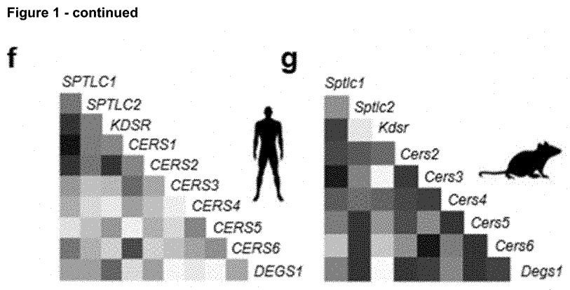

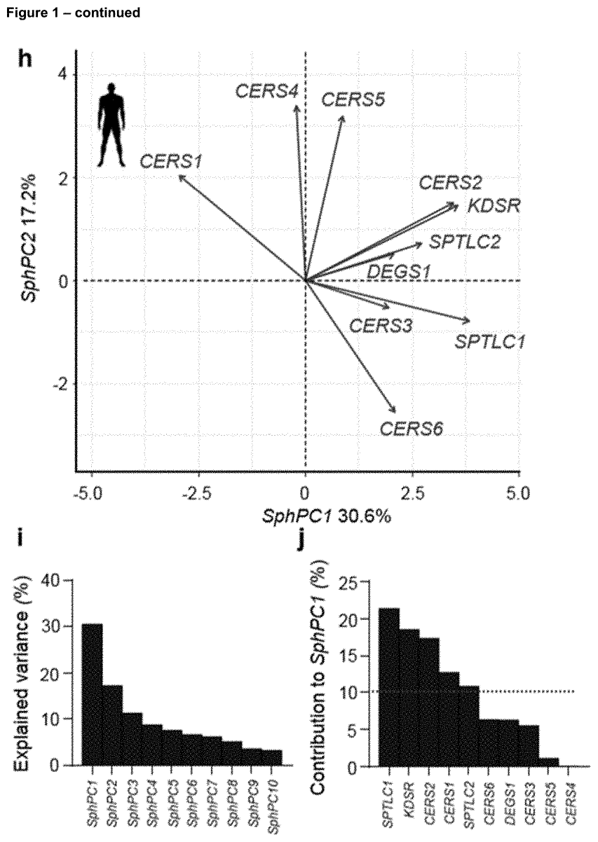

FIG. 1. Sphingolipid de novo synthesis is activated upon aging in skeletal muscle. (a) Scheme of the sphingolipid de novo synthesis pathway. (b) Transcript abundance of enzymes of sphingolipid de novo synthesis pathway in human skeletal muscle from individuals in the Gene-Tissue Expression (GTEx) project (n=491). (c) Total ceramide levels in liver, brain, skeletal muscle, and plasma of young (8-week old, n=10) and aged (24-month-old, n=10) C57BL/6JRj mice. (d) Concentrations of individual ceramide species in mouse quadriceps muscle. (e) Transcript abundance of enzymes of sphingolipid de novo synthesis pathway in skeletal muscle of young and aged individuals. (f) Correlation of transcripts of sphingolipid de novo synthesis pathway in human skeletal muscle (GTEx, n=491). (g) Correlation of transcripts of sphingolipid de novo synthesis pathway in skeletal muscle of 42 genetically diverse BXD strains. (h) A factor loading plot (biplot) showing the effects of the enzymes of sphingolipid de novo synthesis pathway on two first principal components (SphPC1 and SphPC2) in human skeletal muscle (GTEx). (i) Proportion of variance explained by each principal component of the sphingolipid de novo synthesis pathway. (j) Contribution of each transcript to the SphPC1 in human skeletal muscle (GTEx). (k) Correlation of SphPC1 in skeletal muscle with measurements of muscle mass and function in BXD mice. (l) Correlation of Sptlc1 in skeletal muscle with measurements of muscle mass and function in BXD mice. All data are shown mean±SEM. *P<0.05, **P<0.01, and ***P<0.001 with Student's two-tailed T test.

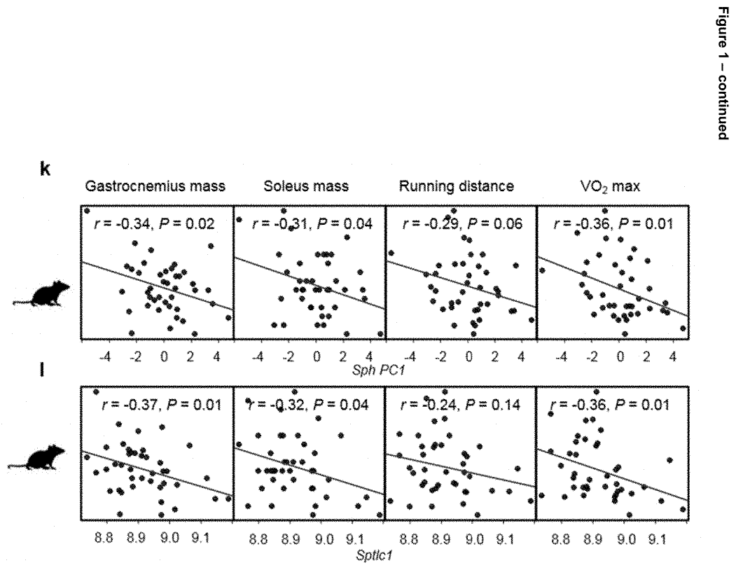

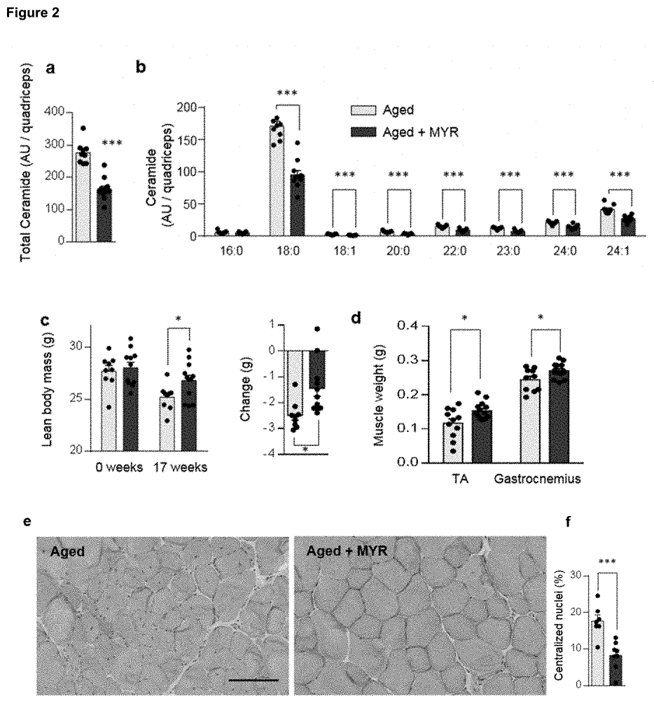

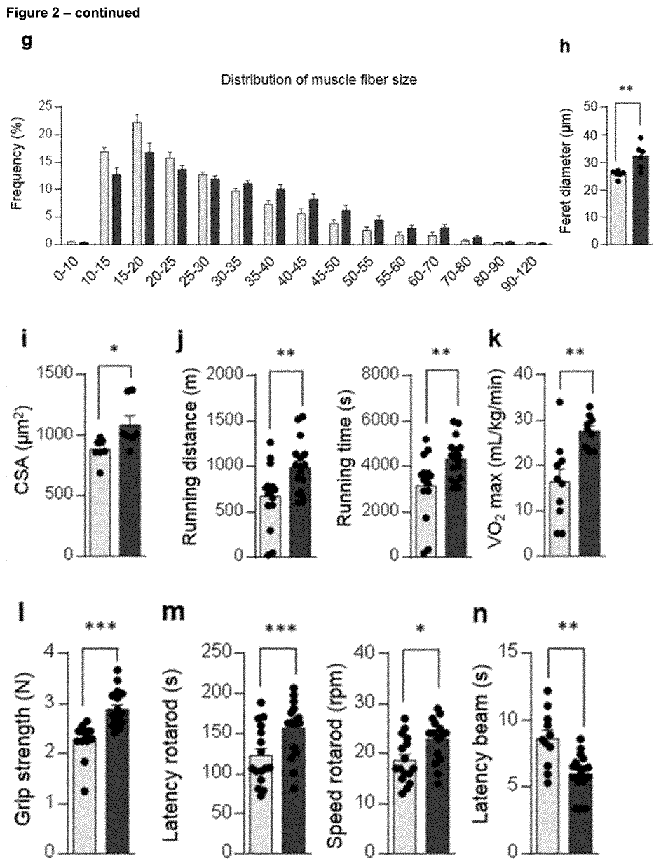

FIG. 2. Inactivation of sphingolipid de novo synthesis increases muscle mass and improves muscle function. Aged (18-month-old) C57BL/6JRj mice were treated with intraperitoneal injections of myriocin (MYR) for 5 months @ 0.4 mg/kg/3 times per week. (a) Total ceramide levels in liver, brain, skeletal muscle, and plasma. (b) Concentrations of individual ceramide species in mouse quadriceps muscle. (c) Lean body mass measured before and after treatment, and its change. (d) Gastrocnemius and tibialis anterior (TA) mass. (e) Hematoxylin and eosin (H&E) staining of TA muscle. Scale bar, 50 μm. (f) Proportion of fibers with centralized nuclei. Distribution (g), mean of fiber minimal Feret diameter (h), and cross-sectional area (CSA) (i) in TA muscle. Comparison of maximal running distance and duration (j), aerobic capacity (k), grip strength (l), latency and maximal speed of rotarod test (m), and latency of beam crossing (n) between aged DMSO and myriocin treated mice. For all experiments, n=6-12 per group. All data are shown mean±SEM. *P<0.05, **P<0.01, and ***P<0.001 with Student's two-tailed T test.

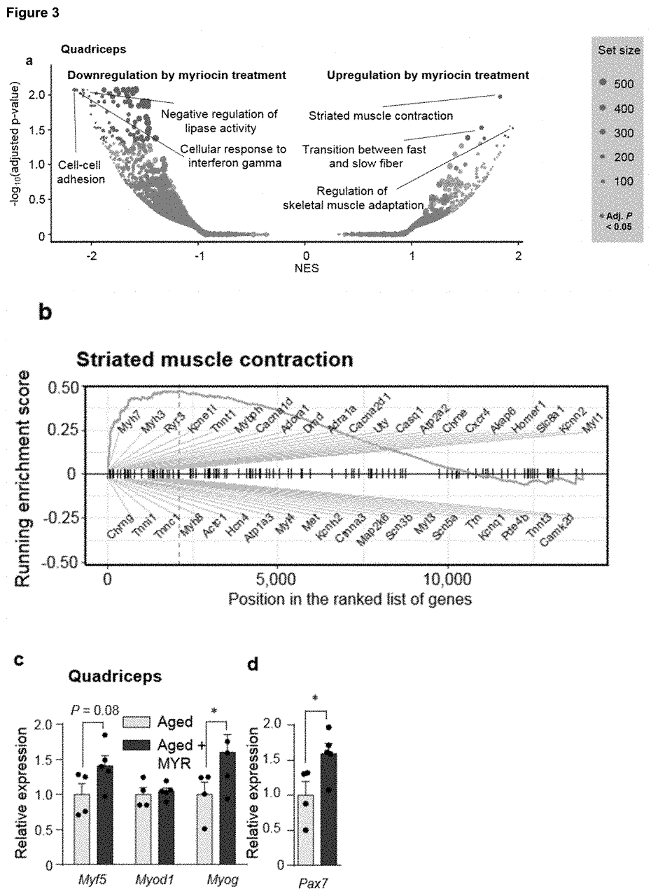

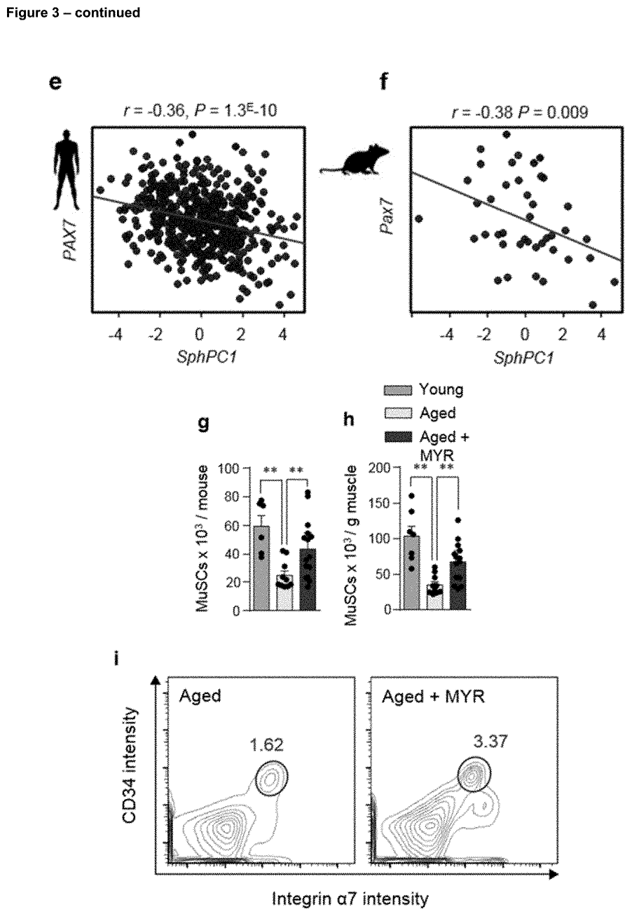

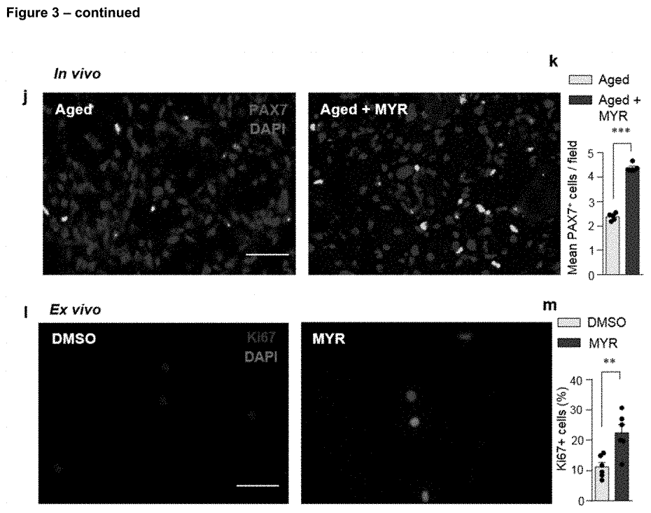

FIG. 3. Inhibition sphingolipid de novo synthesis increases MuSC proliferation and tissue count. RNA-seq transcriptome analysis from quadriceps muscle of aged mice treated with DMSO or myriocin. Volcano plot (a) of the genesets (GO categories) with nomalized enrichment score (NES) and adjusted p-value for each geneset given. Enrichment plot of the ‘Striated muscle contraction’ (b), the most upregulated GO category by myriocin. Comparison of transcripts of Myf5, Myod1, and Myog (c), and the MuSC marker Pax7 (d). (e) Correlation of SphPC1, i.e. the first principal component of the sphingolipid de novo synthesis pathway, with PAX7 in human skeletal muscle (GTEx, n=491). (f) Correlation of SphPC1 with Pax7 in mouse skeletal muscle (BXD, n=42). Number of freshly isolated MuSCs from total hindlimbs musculature (g), normalized to muscle weight (h) from young, aged, and aged mice treated with myriocin. (i) FACS contour plot of a7 integrinCD34Sca-1-CD45-CD31-CD11b-cells which correspond to MuSCs isolated from aged and aged mice treated with myriocin. Representative images (j) and quantification (k) of PAX7-immunostained cells in TA muscle. DAPI, 4′6-diamino-2-phenylindole. Scale bar, 50 μm. Immunocytochemistry (l) and quantification (m) of Ki67 positive cells from freshly isolated MuSCs from aged C57BL/6JRj mice after 72 h incubation in DMSO or myriocin containing medium. Scale bar, 50 μm. All data are shown mean±SEM. *P<0.05, **P<0.01, and ***P<0.001 with Student's two-tailed T test.

FIG. 4. Sphingolipid depletion improves MuSC function and regenerative capacity in vivo. (a) Aged C57BL/6JRj mice were treated with myriocin, and upon sacrifice, freshly isolated MuSCs were transplanted into the TA of either cartdiotoxin damaged aged C57BL/6JRj or mdx recipient mice. Representative images and quantification of PAX7 (b-c) and dystrophin (d-e) immunostained TA muscle from mdx recipients 7 days after transplantation. Scale bar, 50 μm. Representative images and quantification of PAX7 (f-g) and embryonic myosin heavy chain (eMyHC) (h-i) immunostained TA muscle from aged C57BL/6JRj mice at 7 days after transplantation. Scale bar, 50 μm in PAX7 staining. Scale bar, 250 μm in eMyHC staining. Representative images and quantification of H&E stained TA from aged C57BL/6JRj recipients at 7 days (j-k) and 14 days (I-m) after transplantation. Scale bar, 50 μm. Maximal running distance (n), grip strength (o), and latency on a rotarod (p) before and 7 days after transplantation. The change relative to baseline is reported. All data are shown mean±SEM. *P<0.05, **P<0.01, and ***P<0.001 with Student's two-tailed T test.

FIG. 5. Sphingolipid depletion cell-autonomously activates muscle differentiation program in MuSCs and myoblasts. (a) Immunocytochemistry and quantification of MYOD from freshly isolated MuSCs from aged C57BL/6JRj mice after 72 h incubation in DMSO or myriocin containing medium. (b) Immunocytochemistry and quantification of MYOG from freshly isolated MuSCs from aged C57BL/6JRj mice. Immunocytochemistry (c), fusion index (d), myotube area (e), and expression of myogenesis markers (f) from C2C12 myoblasts grown in DMSO and myriocin containing medium. Immunocytochemistry (g), fusion index (h), myotube area (i), and gene expression of myogenesis markers (j) from C2C12 myoblasts silenced for Sptlc1 using CRISPR-Cas9. Immunocytochemistry (k), fusion index (l), myotube area (m), and gene expression of myogenesis markers (n) from C2C12 myoblasts silenced for Cers2 using CRISPR-Cas9. All data are shown mean±SEM. *P<0.05, **P<0.01, and ***P<0.001 with Student's two-tailed T test. Scale bar, 50 μm.

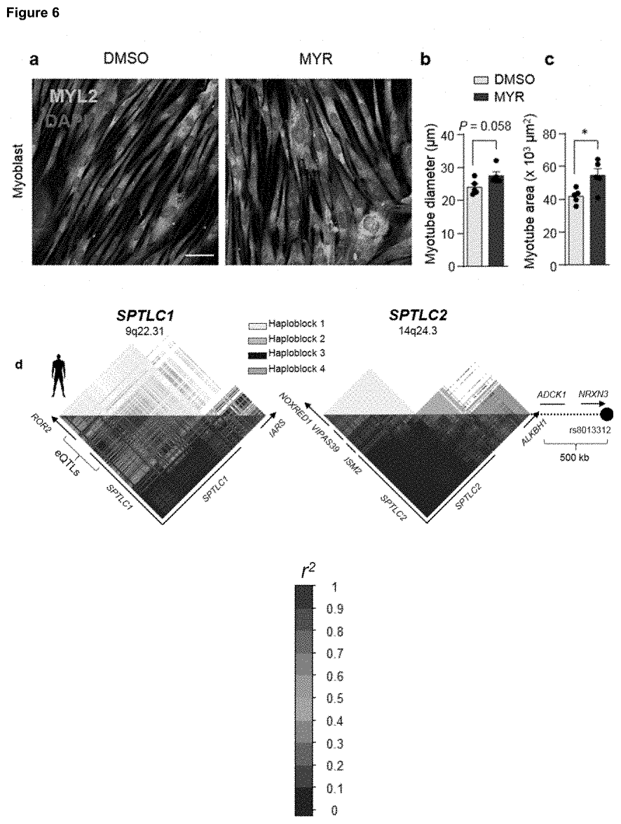

FIG. 6. Genetic evidence points to beneficial effects of sphingolipid depletion in humans. Immunocytochemistry (a), myotube diameter (b), and myotube area (c) of human primary myoblasts treated with DMSO or myriocin. All data are shown mean±SEM. *P<0.05 with Student's two-tailed T test. Scale bar, 50 μm. Haploblock structure of human SPTLC1 (d) and SPTLC2 (e) loci. (f) Associations of SPTLC1 and SPTLC2 cis-eQTLs rs10820917 and rs8013312 with mRNA levels of SPTLC1 and SPTL2 in GTEx, respectively, and senior fitness test (SFT) score, and its component traits in Helsinki Birth Cohort Study. The forest plot represents 3 with 95% confidence interval (CI). (g) Violin plots of skeletal muscle mRNA, SFT score, and distance in a 6-min walking test as a function of SPTLC1 and SPTLC2 cis-eQTLs rs10820917 and rs8013312, respectively.

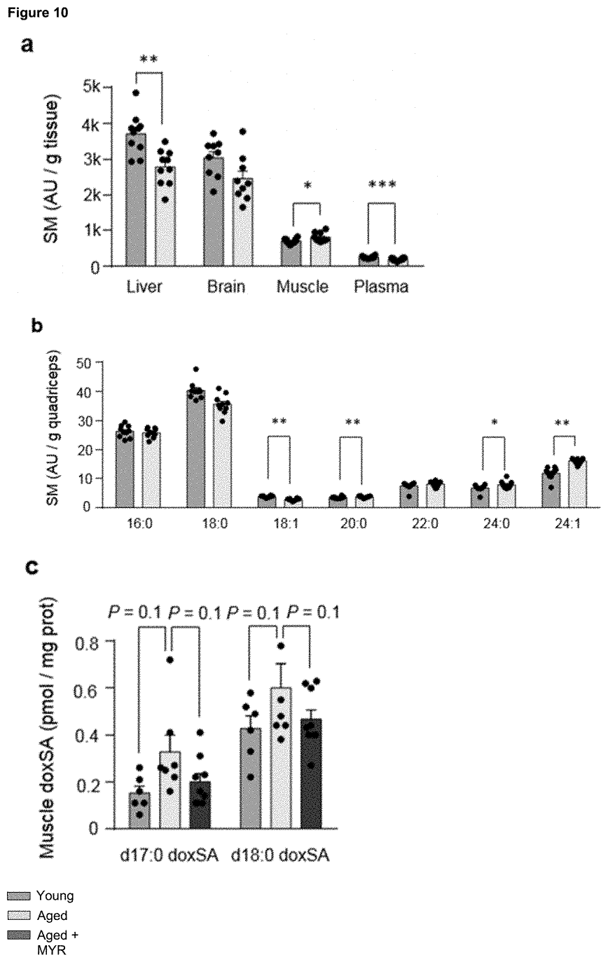

FIG. 7. Levels of Sphinganine (SA) (a) and sphingosine (SO) (b) in quadriceps of young mice, aged mice, and aged mice treated with myriocin. See FIG. 10 for details.

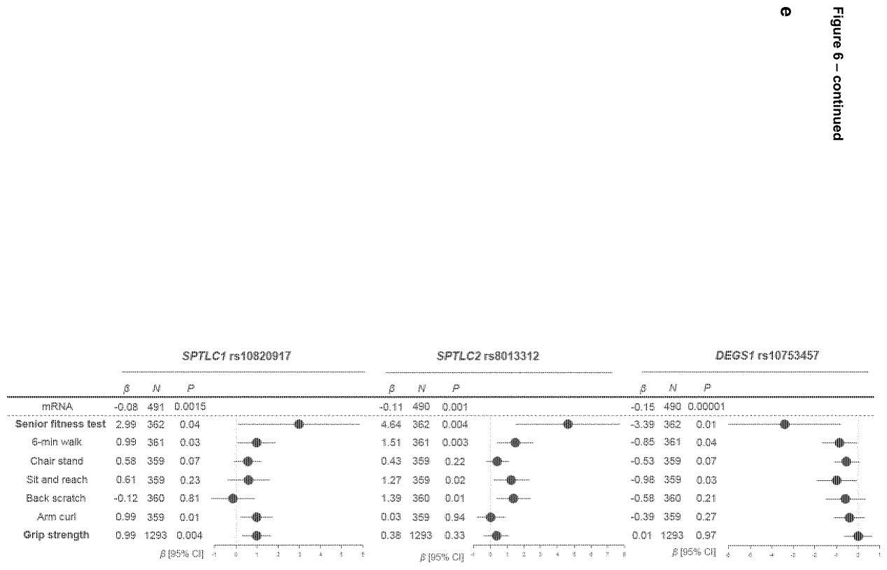

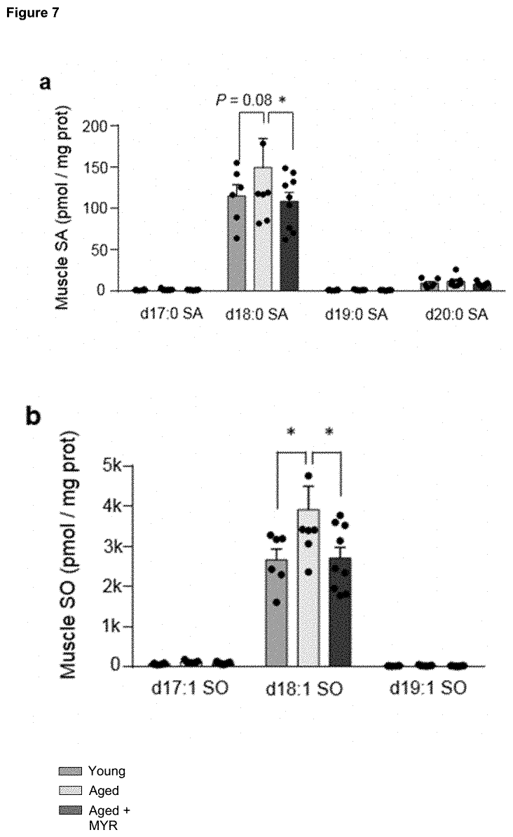

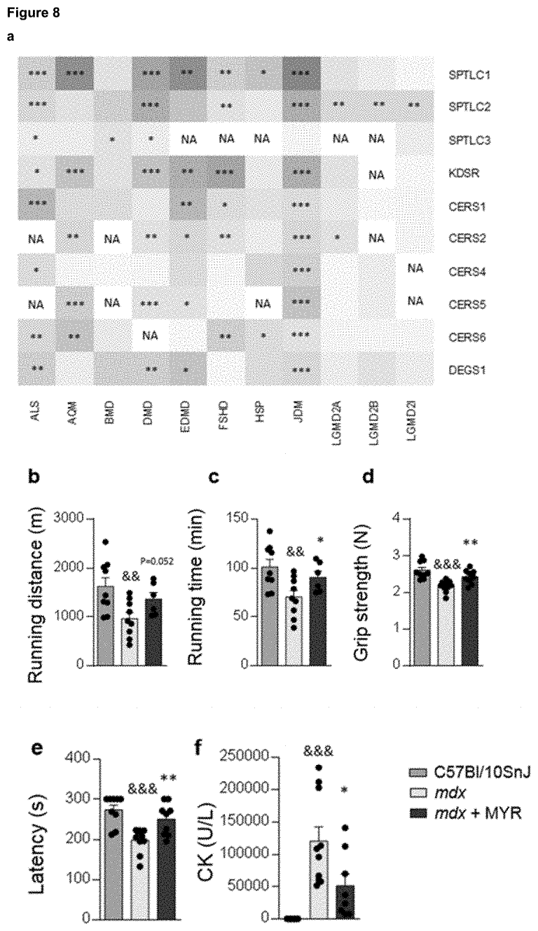

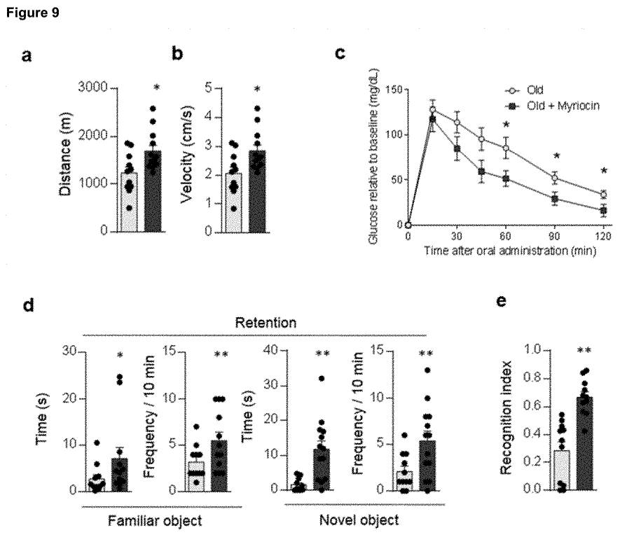

FIG. 8. Sphingolipid inhibition counteracts Duchenne muscular dystrophy. (a) Transcripts of the enzymes of sphingolipid biosynthetic pathway are upregulated in skeletal muscle of patients with muscular dystrophies. Running distance (b), running time (c), grip strength (d), latency on a rotarod (e), and plasma creatine kinase (f) after down-hill running in mdx mice treated with myriocin. All data are shown mean±SEM. *P<0.05, **P<0.01, and ***P<0.001 with Student's two-tailed T test.$

FIG. 9. Sphingolipid depletion protects against frailty phenotypes. (a) Distance moved in 10 mins. (b) Average velocity during 10 minutes of observation. (c) Glucose tolerance test. (d) Time exploring familiar and novel objects. (e) Recognition index. Aged (18-month-old) C57BL/6JRj mice were treated with intraperitoneal injections of myriocin (MYR) for 5 months @ 0.4 mg/kg/3 times per week.

FIG. 10. Sphingomyelin and deoxysphingolipid levels in skeletal muscle in aging and upon myriocin treatment. Concentrations of total sphingomyelin (SM) in liver, brain, skeletal muscle, and plasma of young (8-week old, n=10) and aged (24-month-old, n=10) C57BL/6JRj mice (a), and concentrations of individual sphingomyelin species in quadriceps of young and aged (b) C57BL/6JRj mice. Concentrations of 1-deoxysphinganine (c) in quadriceps muscle of young, aged, and aged mice treated with myriocin. (d) A factor loading plot (biplot) showing the effects of the enzymes of sphingolipid de novo synthesis pathway on two first principal components (SphPC1 and SphPC2) in BXD mouse skeletal muscle. (e) Proportion of variance explained by each principal component of the sphingolipid de novo synthesis pathway. (f) Contribution of each transcript to the SphPC1 in mouse skeletal muscle (BXD). Concentrations of total sphingomyelin in liver, brain, muscle, and plasma (g), and individual sphingomyelin species (h) in quadriceps muscle of aged mice and aged mice treated with myriocin. All data are shown mean±SEM. n=6-10 per group. *P<0.05, **P<0.01, and ***P<0.001 with Student's two-tailed T test.

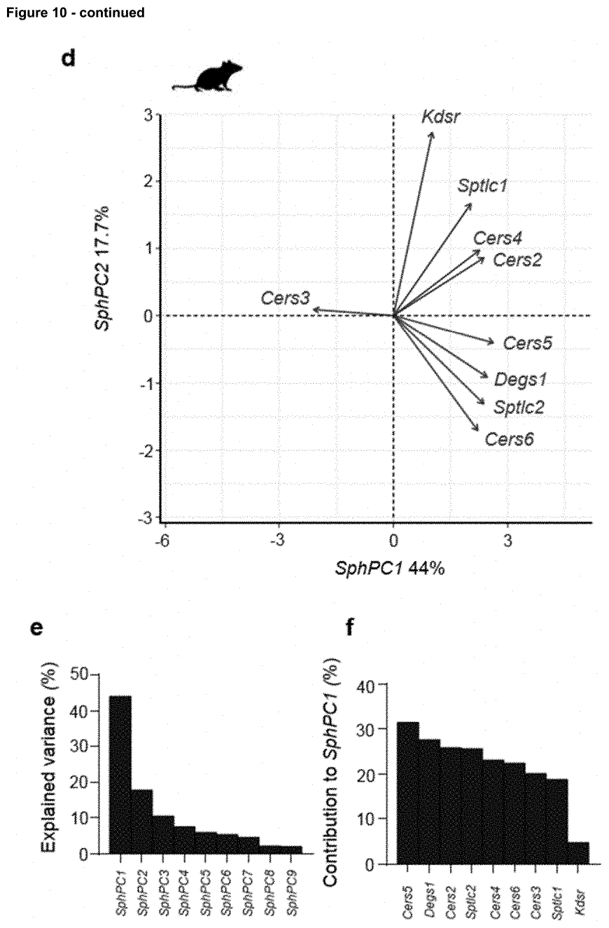

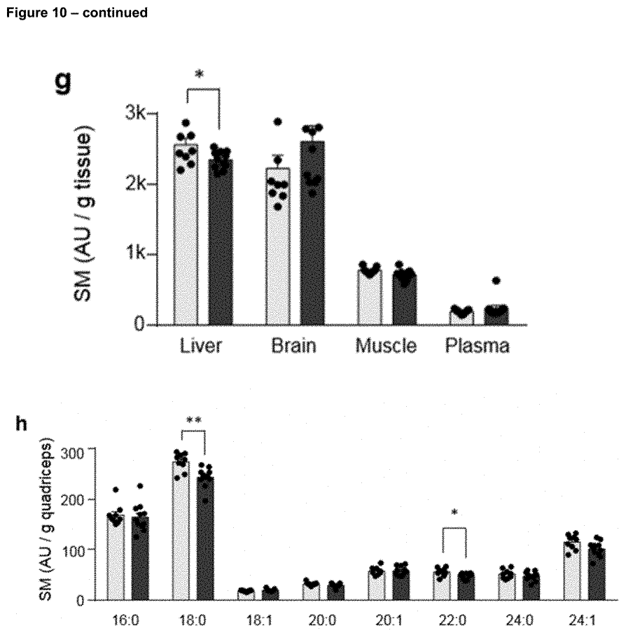

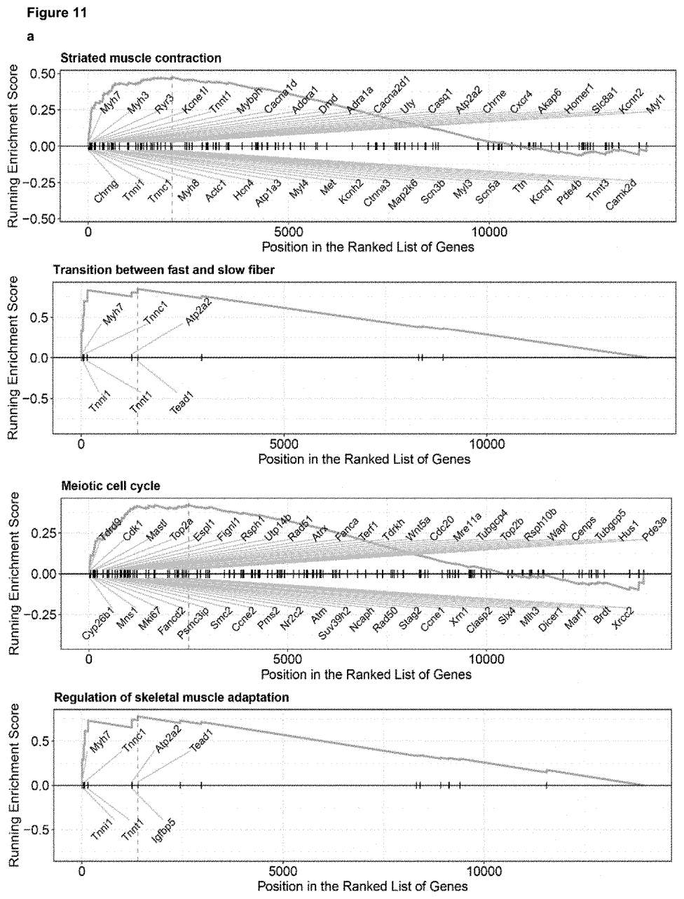

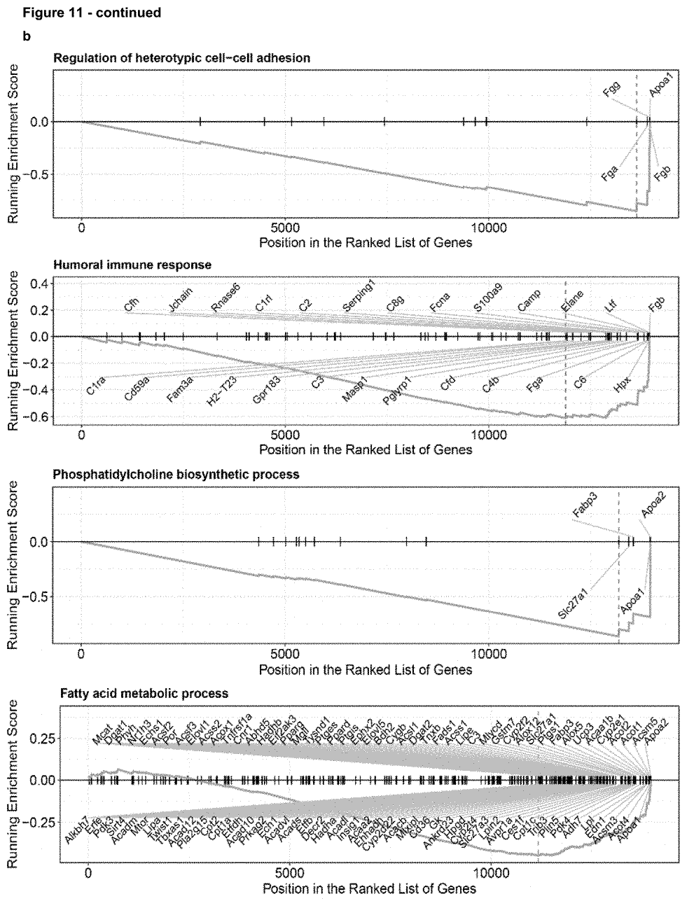

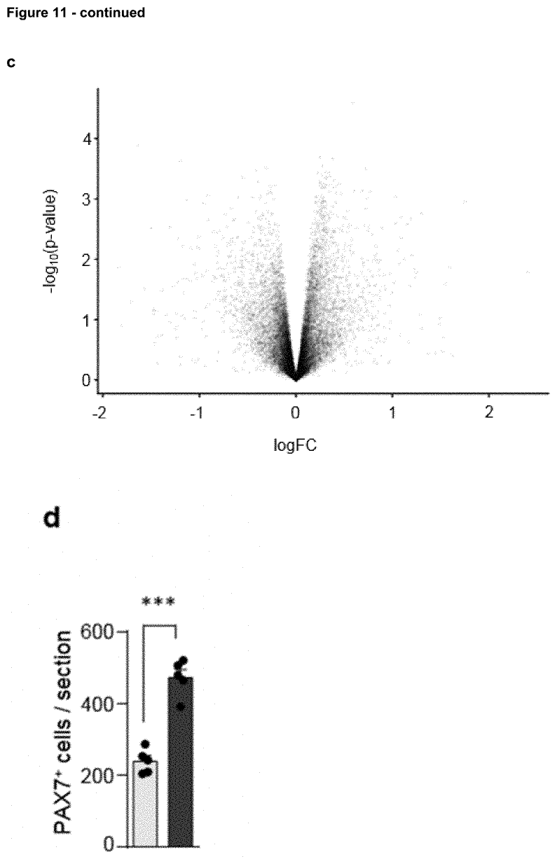

FIG. 11. Inhibition sphingolipid de novo synthesis increases MuSC proliferation and tissue count. Upregulated (a) and downregulated (b) GO categories in quadriceps muscle of aged C57BL/6JRj mice with myriocin started at 18 months of age in RNA sequencing. (c) Volcano plot of individual genes displaying log of nominal p-value (vertical axis) and log₂ fold change (horizontal axis) in quadriceps of myriocin treated mice. (d) Quantification of PAX7 positive cells per section in quadriceps muscle. All data are shown mean±SEM. ***P<0.001 with Student's two-tailed T test.

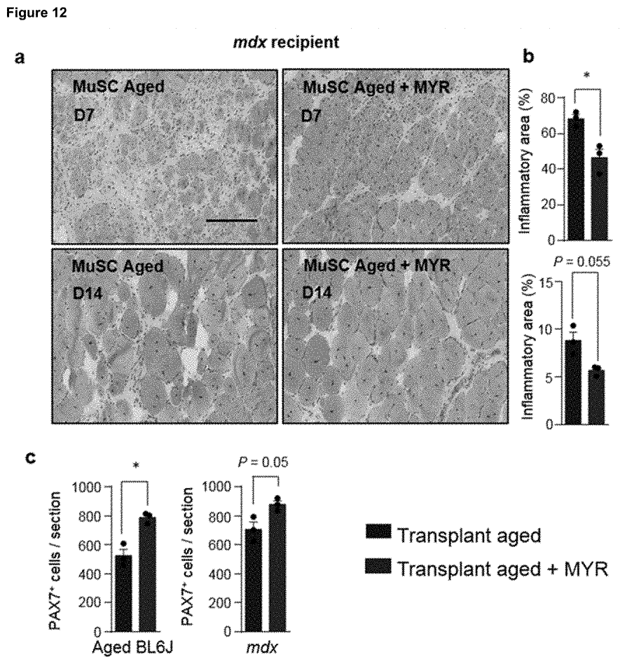

FIG. 12. Sphingolipid depletion improves MuSC function and muscle regeneration in vivo. Aged 18-month-old C57BL/6JRj mice were treated with myriocin, and upon sacrifice, freshly isolated MuSCs were transplanted to either aged C57BL/6JRj or mdx mice. Representative images (a) and quantification of inflammatory area (b) of H&E stained TA from mdx recipients at 7 days and 14 days after transplantation. Scale bar, 50 μm. (c) Quantification of PAX 7 positive cells per section in aged C57BL/6JRj and mdx recipient mice. All data are shown mean±SEM. *P<0.05, **P<0.01, and ***P<0.001 with Student's two-tailed T test.

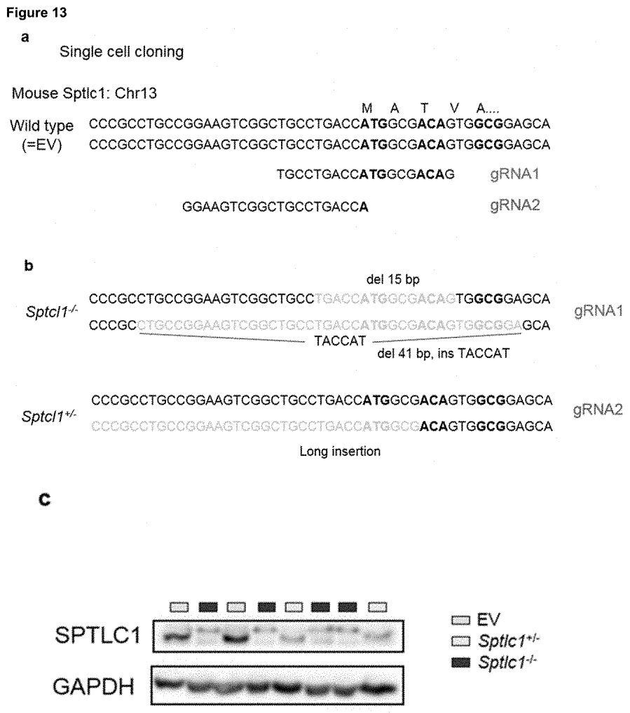

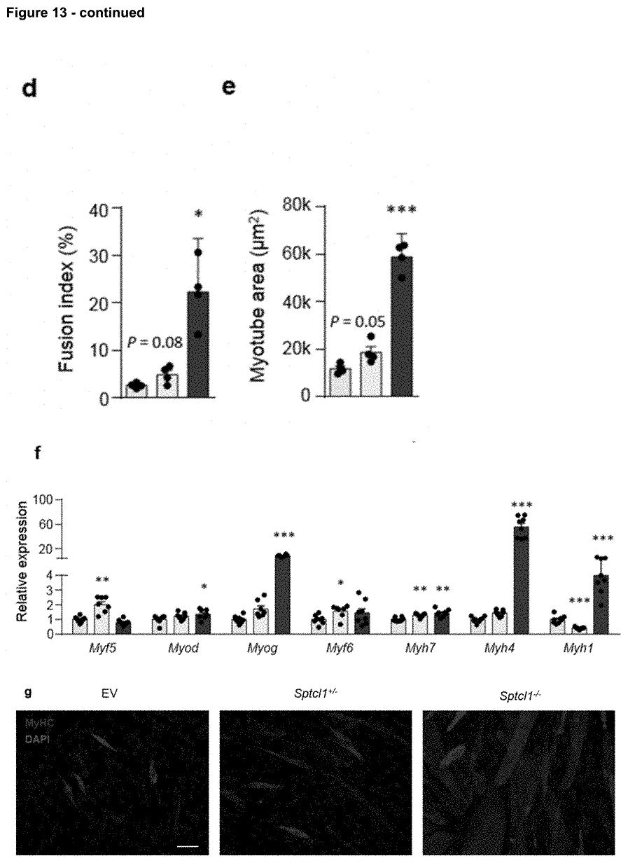

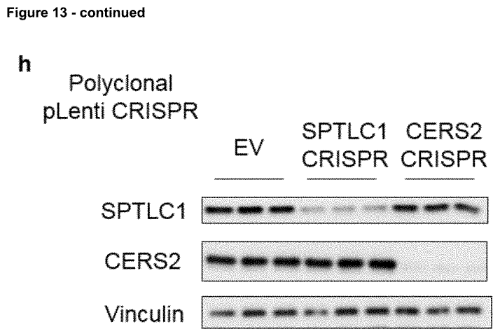

FIG. 13. Single clone CRISPR mediated Sptlc1 knockout promotes muscle cell differentiation in C2C12 myoblasts in a dose-dependent manner. (a) Location guide RNA (gRNA) binding sites in Sptlc1 for two different gRNAs, gRNA1 and gRNA2. (b) Changes in DNA induced by gRNA1 and gRNA2 in C2C12 myoblasts. gRNA1 transfection induced deletions in two homologous chromosomes resulting homozygous Sptlc1 knockout (Sptlc1^{-/-}). gRNA2 induced a long insertion in one homologous chromosome leaving the other chromosome intact, resulting in heterozygous knockout (Sptlc1^{+/-}). (c) Verification of Sptlc1^{+/-} and Sptlc1^{-/-} knockout C2C12 cell lines using Western blot. Fusion index (d), myotube diameter (e), transcript expression of myogenesis markers (f), and immunocytochemistry (g) from C2C12 myoblasts with heterozygous and homozygous loss-of-function of Sptlc1 using single clone CRISPR-Cas9. Scale bar, 50 μm. (h) Polyclonal silencing of Sptlc1 and Cers2. Verification of knockouts using Western blot. All data are shown mean±SEM. *P<0.05, **P<0.01, and ***P<0.001 with Student's two-tailed T test.

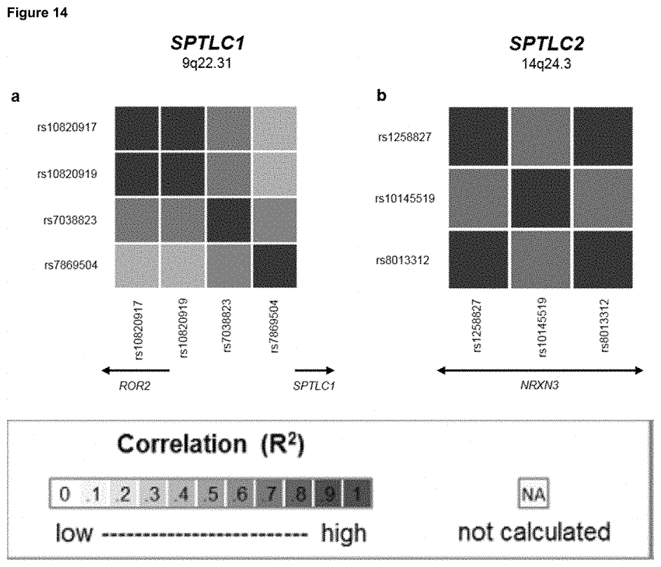

FIG. 14. Linkage disequilibrium (LD) structure of cis-eQTL of human SPTLC1 and SPTLC2. (a) LD structure of SPTLC1 (a) and SPTLC2 (b) cis-eQTLs denoted by r² measurements rom Phase 3 (Version 5) of the 1000 Genomes Project.

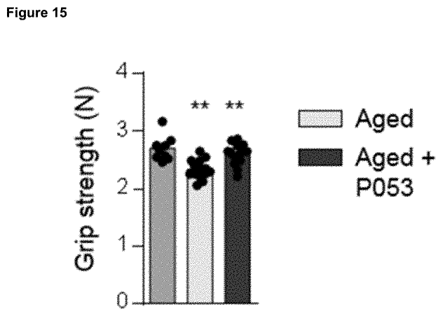

FIG. 15. Inhibition of ceramide synthase improves grip strength upon aging. Aged C57BL/6JRj male mice (20 months of age) were treated with P053, an inhibitor of ceramide synthase suitable for oral administration. P053 recovered the loss in grip strength seen in aged mice.

FIG. 16. Quantitative Proteostat signal normalized over the number of cells in human primary myoblasts from IBM donors (above) and APPSwe-expressing C2C12 myoblasts (below) decreases after treatment with 30 μM MYR for 24 h. Values are expressed as mean±s.e.m. ***P:0.001; ****P:0.0001. Differences between two groups were assessed using Student's two-tailed t-tests (error bars: 95% confidence intervals).

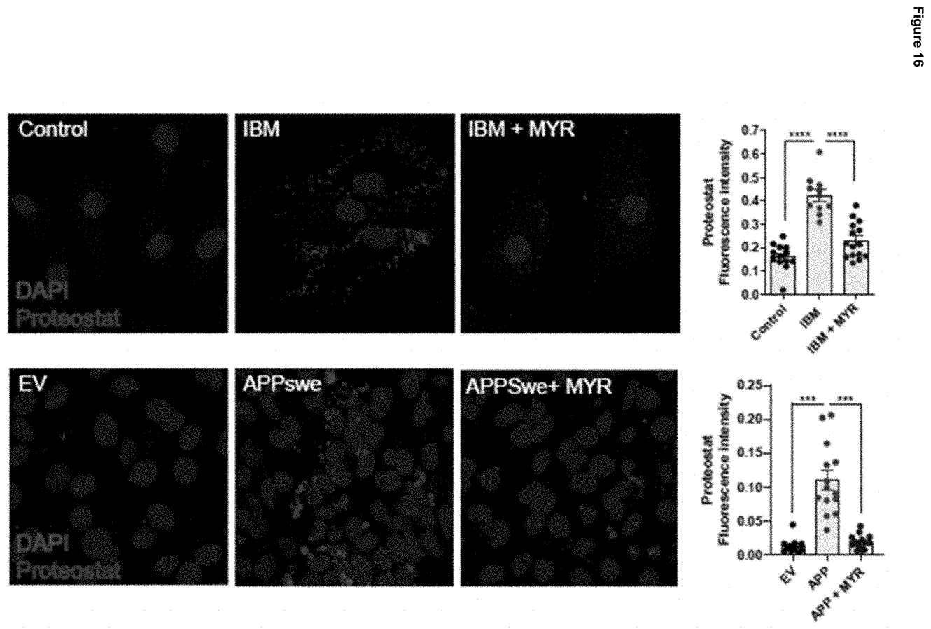

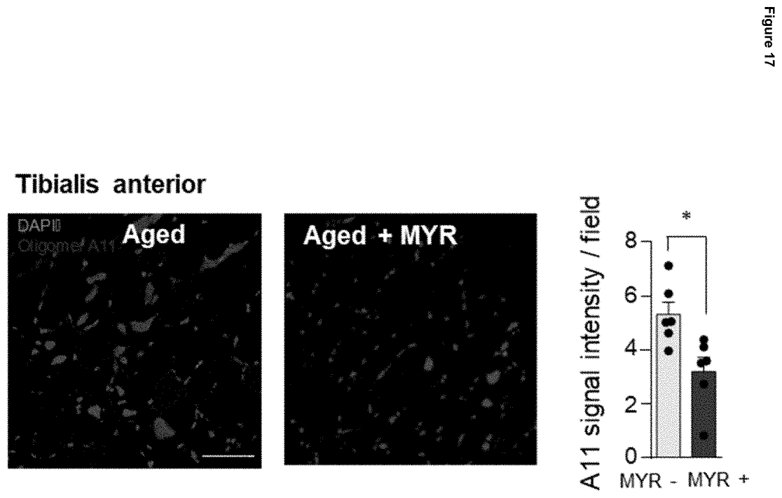

FIG. 17. Myriocin improves proteostasis in aged mice. Values are expressed as mean±s.e.m. *P≤0.05. Differences between two groups were assessed using Student's two-tailed t-tests (error bars: 95% confidence intervals).

The examples illustrate the invention.

### EXAMPLE 1—CERAMIDES ACCUMULATE IN SKELETAL MUSCLE UPON AGING

Sphingolipid de novo synthesis pathway produces ceramides and other sphingolipids by using fatty acids and amino acids as substrates (FIG. 1a). SPT converts L-serine and palmitoyl-CoA to 3-ketosphinganine, which is rapidly converted to sphinganine. Coupling of sphinganine to long-chain fatty acid is accomplished by one of 6 distinct mammalian ceramide synthases of which CERS2 is the most abundant in skeletal muscle (FIG. 1b). To study how aging affects the activity of sphingolipid de novo synthesis pathway in skeletal muscle in vivo, total ceramide content of different organs in young (2-month-old) and aged (2-year-old) mice was compared. We observed an increase in skeletal muscle ceramides was observed (FIG. 1c). Here the focus was on skeletal muscle sphingolipids and potential benefits upon their reduction.

The trend of age-dependent increase in skeletal muscle ceramides was global, comprising ceramide species with different acyl lengths (FIG. 1d). The most pronounced increase upon aging was observed for Cer(d18:1/24:1), and the most abundant ceramide in muscle, Cer(d18:1/18:0), displayed a 20% increase upon aging (FIG. 1d). Differences in skeletal muscle sphingomyelin (SM), a sphingolipid consisting of phosphocholine and ceramide, were less consistent between young and aged mice (FIG. 10a-b). Deoxysphingolipids are synthesized by SPT through conjugation of L-alanine rather than L-serine with fatty acid, and have been implicated in disease conditions, such as diabetes. Similar to canonical sphingolipids, there was a trend of deoxysphinganine (doxSA) accumulation in skeletal muscle upon aging (FIG. 10c).

Transcript abundance of the enzymes of sphingolipid de novo biosynthesis pathway between young and aged human individuals in a publicly available dataset (GSE25941) was next compared. Consistent with increased muscle ceramides, many transcripts of these enzymes were upregulated upon aging, including SPTLC1, KDSR, CERS2, and CERS5 (FIG. 1e). The only down-regulated enzyme was CERS1 (FIG. 1e) whose expression is relatively low in skeletal muscle in human Genotype-Tissue Expression (GTEx) database (FIG. 1B). In general, there was a strong positive correlation between different transcripts of the sphingolipid de novo biosynthesis pathway in post-mortem skeletal muscle biopsies in human GTEx dataset (n=491) (FIG. 1f). Only CERS1 transcript displayed a negative correlation with the other enzymes. Similarly, in the mouse BXD strains, a recombinant inbred mouse population with substantial genetic heterogeneity, the transcripts were directly correlated with each other (FIG. 1g). These results suggest that sphingolipid de novo biosynthesis pathway is under tightly coordinated transcriptional control.

Following the strong correlations between transcripts of the pathway, principal component analysis of the sphingolipid de novo biosynthesis pathway in humans (FIG. 1h) and mice (FIG. 10d-f) was conducted. The first principal component of the expression of different enzymes of the sphingolipid de novo synthesis pathway (SphPC1) explained 30.6% of the variance in human skeletal muscle expression (FIG. 1i). Of the transcripts of the pathway, Sptlc1 contributed the most, accounting for 21% of SphPC1 variability (FIG. 1j). In BXD mouse reference population correlations were examined between SphPC1 and different parameters of muscular fitness, including measures of muscle mass and function, components of sarcopenia. Negative correlation was observed between SphPC1 and both gastrocnemius and soleus muscle masses, as well as performance on a treadmill and maximal aerobic capacity (FIG. 1k). Sptlc1 skeletal muscle expression showed similar correlations with these phenotypes (FIG. 1l). These findings demonstrate that the expression of sphingolipid de novo biosynthesis is inversely correlated with muscle mass and function, and suggest its involvement in sarcopenia.

### EXAMPLE 2—INHIBITION OF SPHINGOLIPID DE NOVO SYNTHESIS PREVENTS LOSS OF MUSCLE MASS AND FUNCTION IN AGING

To examine whether a causal relationship could underlie the correlation between SphPC1 and muscle mass and function, it was tested whether inhibition of sphingolipid de novo biosynthesis pathway could protect against age-related muscle dysfunction. Aged (18-month-old) C57Bl/6JRj mice were treated for 5 months on chow diet with myriocin, a specific inhibitor of SPT, the first and rate-limiting enzyme of the sphingolipid de novo synthesis pathway, and whose expression in skeletal muscle was negatively correlated with muscular fitness in BXDs. Myriocin treatment reduced total ceramide contents of skeletal muscle (FIG. 2a) as well as individual ceramide species in a global fashion (FIG. 2b), confirming the efficacy of the compound in skeletal muscle. In addition to ceramides, myriocin treatment reduced skeletal muscle deoxysphingolipid contents, yet with smaller effect than L-serine derived canonical sphingolipids (FIG. 10c).

Importantly, myriocin treatment improved body composition of aged mice. Myriocin delayed age-dependent decline in lean body mass (FIG. 2c), and increased muscle mass. Myriocin treated mice displayed greater gastrocnemius and TA mass than DMSO treated controls (FIG. 2d). Improved muscle morphology was evident in histological analysis of tibialis anterior (TA) muscles of myriocin treated mice (FIG. 2e), manifested by reduced number of centralized nuclei (FIG. 2f), a hallmark of muscle aging, and larger cross-sectional area of muscle fibers (FIG. 2g-i). Myriocin treatment also counteracted age-related muscle dysfunction. Aged mice treated with myriocin demonstrated improved exercise performance and muscle strength, evidenced by the increased running distance and time on a treadmill (FIG. 2j), improved aerobic capacity (FIG. 2k), and grip strength (FIG. 2l). Myriocin treated mice also displayed better muscle coordination, as shown by their improved performance in the rotarod test (FIG. 2m), and faster crossing of a beam (FIG. 2n). Overall, myriocin treatment improved muscle morphology and counteracted age-related loss of muscle mass, strength, endurance, and coordination, indicating protection against age-related sarcopenia.

### EXAMPLE 3—SPHINGOLIPID DEPLETION ENHANCES PROLIFERATIVE CAPACITY OF MUSCLE STEM CELLS

To identify biological pathways whose modulation could explain improved muscle function upon myriocin treatment, transcriptomes of quadriceps muscle of aged myriocin and DMSO treated mice using RNA sequencing were compared. Gene set enrichment analysis (GSEA) ranking transcripts based on their fold change were performed. The most downregulated pathways by myriocin treatment were related to the extensively studied role of sphingolipids in lipid metabolism and inflammation, while upregulated pathways were related to muscle contraction and differentiation (FIG. 3a-b, 11a-c), suggesting improved regeneration. Indeed, in a targeted analysis of classical transcription factors regulating myogenesis, Myog and Myf5 were upregulated in skeletal muscle upon myriocin treatment (FIG. 3c).

Muscle stem cells (MuSCs) are capable of giving rise to mature muscle fibers, and their regenerative capacity has been reported to be impaired upon aging. The expression of Pax7, a specific marker of MuSCs, was upregulated in the quadriceps muscle of myriocin treated mice (FIG. 3d). Consistent with this, PAX7 expression correlated negatively with the first PC of the ceramide synthetic pathway in both human (FIG. 3e) and mouse (FIG. 3f) skeletal muscle. To study whether increased Pax7 transcript abundance in skeletal muscle reflects elevated MuSC count, a7 integrin/CD34 double positive cells were isolated from the hind-limbs using fluorescence-activated cell sorting (FACS). Consistent with previous reports the number of MuSCs was decreased in hindlimbs of aged mice as compared to young mice, and myriocin treatment restored the MuSC pool of aged mice close to that of young mice (FIG. 3g-i). Tissue sections of TA from myriocin treated mice revealed elevated number of PAX7⁺ muscle cells (FIG. 3j-k, FIG. 11d), suggesting increased MuSC proliferation, and MuSCs isolated from untreated aged mice and cultured ex vivo in myriocin containing medium resulted in higher Ki67⁺ MuSC count (FIG. 3l-m), indicating that myriocin treatment enhances MuSC proliferation. Upon aging, there is a decline in self-renewal and proliferative potential of MuSCs, and our findings suggest that sphingolipid depletion could counteract these defects.

### EXAMPLE 4—MYRIOCIN PRIMES MUSCS FOR ACCELERATED REGENERATION

Decline in tissue regenerative potential is a major hallmark of mammalian aging, including skeletal muscle. It was next asked the question whether myriocin treatment induces functional improvement of MuSCs, boosting their regenerative capacity. MuSCs from aged mice treated or untreated with myriocin for 5 months were isolated, and transplanted into tibialis anterior (TA) muscles of recipient mice. The recipient mice were either aged (20-month-old) C57Bl/6JRj or aged (1-year-old) mdx mice, a mouse model of Duchenne muscular dystrophy lacking dystrophin protein (FIG. 4a). Before MuSC transplantation, injury was induced by cardiotoxin injection to TA of all recipient mice. After 7 days of transplantation, mdx mice displayed elevated count of PAX7⁺ cells, indicating more efficient MuSC proliferation (FIGS. 4b-c and 12c). MuSCs isolated from myriocin treated mice stimulated myogenesis of dystrophin-positive fibers, verifying the functionality of newly transplanted MuSCs (FIG. 4d-e). At both 7 and 14 days after transplantation the inflammatory area was also smaller (FIG. 12a-b) in mdx recipients of MuSCs isolated from myriocin treated mice. Aged C57Bl/6JRj recipients of MuSCs isolated from myriocin treated mice exhibited similar morphologic changes in muscle to mdx recipients, manifested by increased count of PAX7⁺ cells per field (FIG. 4f-g) and per section (FIG. 12c), higher embryonic myosin heavy chain (eMyHC) positive area (FIG. 4h-i), indicating increased muscle regeneration, and smaller inflammatory area at both 7 and 14 days after injury (FIG. 4j-m). Thus, transplantation of MuSCs derived from myriocin treated donors induce major morphological improvements following cardiotoxin-induced muscle injury.

To test whether the functional improvement of MuSCs upon myriocin treatment also translates into better muscle function of recipient mice, tests of muscle function were performed before and 7 days after MuSC transplantation. After 7 days of MuSC transplantation, aged C57Bl/6JRj mice, which had received MuSCs from myriocin treated mice, displayed improved exercise capacity (FIG. 4n), grip strength (FIG. 4o), and increased latency in rotarod test (FIG. 4p). These findings demonstrate that myriocin-induced improvement in MuSC function also translates into better muscle function.

### EXAMPLE 5—SPHINGOLIPID DEPLETION ACTIVATES MYOGENIC DIFFERENTIATION IN MUSCLE PROGENITOR CELLS

Inhibition of sphingolipid de novo synthesis pathway has previously been linked to metabolic benefits, including improved insulin sensitivity which might affect muscle function. To study whether sphingolipid depletion has a cell-autonomous effect on myogenesis and muscle regeneration, freshly isolated MuSCs in 30 μM myriocin containing culture medium were incubated. Myriocin elevated both MYOD and MYOG protein levels (FIG. 5a-b), indicating that sphingolipid depletion primes MuSCs for myogenic differentiation. Then the effects of myriocin were examined in myoblasts, a later stage myogenic progenitor cell using mouse C2C12 myoblasts. Myriocin accelerated myoblast differentiation (FIG. 5c), as shown by greater fusion index (FIG. 5d) and myotube area (FIG. 5e), and induced a myogenic transcript signature featuring upregulation of myogenic transcription factors, such as Myog, as well markers of mature myotubes, including myosin heavy chain subunits Myh4 and Myh1 (FIG. 5f).

To corroborate the effects of the sphingolipid de novo synthesis pathway on myogenesis, members of the pathway were silenced using polyclonal CRISPR-Cas9 genome editing. Knockout of Sptlc1 induced myoblast differentiation (FIG. 5g), as determined by quantification of fusion index (FIG. 5h), myotube area (FIG. 5i), and myogenic transcript signature similar to myriocin treated cells (FIG. 5j). Also Cers2 was silenced, the most abundant ceramide synthase in skeletal muscle, downstream of SPT. Inactivation of Cers2 led to accelerated myogenesis (FIG. 5k-m), featuring similar gene expression signature to that observed after myriocin treatment (FIG. 5n). Thus, enzymes downstream of SPT are involved in myogenic programming in a cell-autonomous manner.

To further validate our findings, homozygous (Sptlc1^{-/-}) and heterozygous (Sptlc1^{+/-}) single clone knockouts of Sptlc1 were generated using CRISPR-Cas9 in C2C12 myoblasts (FIG. 13a-c). The single clone knockouts promoted myogenesis in allele dose-dependent manner, with Sptlc1^{-/-} myoblasts displaying greater myotube area than Sptlc1^{+/-} or EV cells (FIG. 13d-g). While the polyclonal Sptlc1 and Cers2 knockouts had milder effects on gene expression signature than myriocin (FIG. 5 j,n), the Sptlc1^{-/-} single clone knockouts induced magnitude similar to myriocin (FIG. 13f). Thus, SPT abundance dose-dependently influences muscle differentiation.

### EXAMPLE 6—GENETIC VARIANTS REDUCING SPT EXPRESSION ARE ASSOCIATED WITH IMPROVED FITNESS IN AGED HUMANS

To determine whether sphingolipid depletion could stimulate muscle differentiation in human cell lines, human primary myoblasts were treated with myriocin. Consistent with mouse myoblasts, myriocin accelerated human myoblast differentiation, displaying larger myotube area (FIG. 6a-c). Thus, SPT inhibition could enhance muscle maintenance in human muscles.

It was finally investigated whether the sphingolipid de novo synthesis pathway is involved in age-related muscle dysfunction in humans by gathering evidence from human genetic studies. The objective was to first examine the region near SPTLC1 and SPTLC2 to identify loci that associate with the expression of these genes in skeletal muscle (cis-expression quantitative trait loci (cis-eQTL)), and then test whether these cis-eQTLs are associated with muscular fitness of aged individuals. The regions near SPTLC1 and SPTLC2 were both spanned by 4 haploblocks (r²>0.2) (FIG. 6d). Using skeletal muscle gene expression data from the GTEx project, cis-eQTLs for SPTLC1 and SPTLC2 (Tables 1-2) were identified in tight linkage disequilibrium within the gene (FIG. 14a-b).

TABLE 1
Cis-eQTLs of SPTLC1 in skeletal muscle

| | rs10820917 | | rs10820919 | | rs7869504 | | rs7038823 | |
|---|---|---|---|---|---|---|---|---|
| SPTLC1 | β | P | β | P | β | P | β | P |
| SPTLC1 mRNA | −0.088 | 0.0019 | −0.088 | 0.0019 | −0.084 | 0.0012 | −0.084 | 0.0015 |
| SFT | 2.99 | 0.04 | 2.99 | 0.04 | 0.50 | 0.69 | 1.74 | 0.21 |
| 6-min walk | 0.99 | 0.03 | 0.99 | 0.03 | 0.74 | 0.06 | 1.00 | 0.02 |
| Back scratch | −0.12 | 0.81 | −0.12 | 0.81 | −0.52 | 0.24 | −0.40 | 0.41 |
| Chair stand | 0.58 | 0.076 | 0.58 | 0.077 | 0.21 | 0.44 | 0.31 | 0.31 |
| Sit and reach | 0.61 | 0.23 | 0.61 | 0.24 | 0.06 | 0.89 | 0.25 | 0.60 |
| Arm curl | 0.99 | 0.01 | 0.99 | 0.01 | 0.01 | 0.98 | 0.62 | 0.098 |
| Grip strength | 0.99 | 0.0047 | 0.99 | 0.0046 | 0.33 | 0.29 | 0.84 | 0.0079 |

TABLE 2
Cis-eQTLs of SPTLC2 in skeletal muscle

| | rs8013312 | | rs10145519 | | rs12588277 | |
|---|---|---|---|---|---|---|
| SPTLC2 | β | P | β | P | β | P |
| SPTLC2 | −0.13 | 0.0014 | −0.13 | 0.002 | −0.12 | 0.002 |
| mRNA | | | | | | |
| SFT | 4.64 | 0.0036 | 4.80 | 0.0053 | 4.35 | 0.006 |
| 6-min | 1.51 | 0.0027 | 1.36 | 0.012 | 1.40 | 0.005 |
| walk | | | | | | |
| Back | 1.38 | 0.013 | 1.38 | 0.021 | 1.37 | 0.01 |
| scratch | | | | | | |
| Chair | 0.43 | 0.22 | 0.24 | 0.54 | 0.36 | 0.31 |
| stand | | | | | | |
| Sit and | 1.27 | 0.02 | 1.71 | 0.004 | 1.21 | 0.029 |
| reach | | | | | | |
| Arm | 0.03 | 0.94 | 0.13 | 0.78 | −0.013 | 0.98 |
| curl | | | | | | |
| Grip | 0.38 | 0.33 | 0.63 | 0.13 | 0.48 | 0.22 |
| strength | | | | | | |

The cis-eQTL with the largest effect of SPTLC1, rs10820917, was located within a haploblock spanning 250 kb region between SPTLC1 and its neighboring ROR2 (Haploblock 1) while the most significant cis-eQTL for SPTLC2 was located 680 kb upstream of the gene (FIG. 6d). The major alleles (C) of rs10820917 and rs8013312 were associated with reduced transcript abundance of SPTLC1 and SPTLC2, respectively, in a dose-dependent manner (FIG. 6e-f). They were exclusively associated with SPT transcript levels, and not with any of the neighboring genes (Table 3-4).

TABLE 3
Association of skeletal muscle transcripts of neighboring
genes with SPTLC1 cis-eQTL rs10820917.

| SPTLC1 rs10820917 | NES | P |
|---|---|---|
| ASPN | −0.013 | 0.7 |
| AUH | −0.014 | 0.67 |
| BICD2 | 0.023 | 0.32 |
| CENPP | 0.0043 | 0.93 |
| ECM2 | −0.018 | 0.53 |
| FGD3 | −0.015 | 0.76 |
| IARS | −0.027 | 0.37 |
| IPKK | 0.039 | 0.24 |
| NFIL3 | −0.013 | 0.64 |
| NOL8 | 0.0025 | 0.93 |
| ROR2 | 0.089 | 0.099 |
| SPTLC1 | 0.088 | 0.0019 |
| SUSD3 | −0.0098 | 0.87 |
| ZNF484 | −0.00012 | 1 |

TABLE 4
Association of skeletal muscle transcripts of neighboring
genes with SPTLC2 cis-eQTL rs8013312.

| SPTLC2 rs8013312 | NES | P |
|---|---|---|
| ADCK1 | −0.026 | 0.44 |
| AHSA1 | 0.056 | 0.1 |
| ANGEL1 | 0.0037 | 0.92 |
| GSTZ1 | 0.034 | 0.28 |
| IRF2BPL | −0.0038 | 0.92 |
| NRXN3 | 0.004 | 0.94 |
| SLIRP | −0.016 | 0.45 |
| SNW1 | −0.0019 | 0.92 |
| SPTLC2 | 0.13 | 0.0014 |
| TMED8 | 0.0013 | 0.97 |
| TMEM63C | −0.053 | 0.3 |
| VASH1 | −0.024 | 0.48 |
| VIPAS39 | 0.011 | 0.71 |
| ZDHHC22 | −0.065 | 0.22 |

The SPT protein complex consists of both SPTLC1 and SPTLC2 encoded subunits, and to model the genetic effects of the entire protein complex on its gene expression, a two-locus genetic score was constructed indicating the total number of C alleles within SPTLC1 rs10820917 and SPTLC2 rs8013312 loci. The average gene expression of SPTLC1 and SPTLC2 was dose-dependently associated with the C allele score, individuals homozygous for C allele in both SPTLC1 rs10820917 and SPTLC2 rs8013312 loci displaying the lowest SPTLC1-SPTLC2 expression (FIG. 6e-f).

To test the effects of the identified cis-eQTLs on muscular fitness upon aging, individuals of the Helsinki Birth Cohort Study (N=2,003) were examined, of whom approximately 700 between 70 to 80 years of age underwent both dense marker genotyping and an extensive battery of physical fitness measurements. The objectively measured fitness tests includes arm curl, number of chair stands, chair sit and reach, back scratch, and 6-min walk test, and collectively constitutes the senior fitness test (SFT) score. It was first tested the association of the SPTLC1-SPTLC2 two-locus genetic score with SFT score, and observed that increased C allele count was dose-dependently associated with improved SFT score (P=0.01) (FIG. 6e-f). Of the SFT component traits, 6-min walking distance and chair sit and reach were associated with the SPTLC C allele count (P=0.03 and P=0.04), as well as improved grip strength, a trait not included in SFT test battery (P=0.006) (FIG. 6e). It was then analyzed the most significant cis-eQTL of SPTLC1 and SPTLC2 individually. The reduced expression associated C alleles of rs10820917 and rs8013312 in SPTLC1 and SPTLC2, were associated with higher SFT (P=0.04 and P=0.004) (FIG. 6e-f). Of the component traits, the major C allele of SPTLC1 rs10820917 was associated with improved performance in 6-min walking test (P=0.03), arm curl (P=0.01), as well as grip strength (P=0.004) while the major C allele of SPTLC2 rs8013312 was associated with increased 6-min walking distance (P=0.0003), sit and reach (P=0.02), and back scratch (P=0.01) (FIG. 6e-f). These genetic data, demonstrating that SPT transcript reducing genetic variants improve age-related fitness, are consistent with outcomes from our pharmacological approach in the aged mice, implying that SPT inhibition in human aging could deliver considerable fitness benefits.

### EXAMPLE 7—MYRIOCIN TREATMENT REDUCED SPHINGANINE AND SPHINGOSINE LEVELS IN SKELETAL MUSCLE

Also muscle sphinganine (SA) and sphingosine (SO) levels were measured after myriocin treatment. Aged myriocin treated mice displayed lower levels of SA and SO in skeletal muscle (FIG. 10a-b). These findings indicate that myriocin induces a reduction of every metabolite of the pathway. Thus, inhibiting sphinganine or sphingosine production could confer fitness benefits.

Identification of Dihydroceramides as Suppressors of Age-Related Muscle Dysfunction and Muscle Differentiation

Also Degs1 in C2C12 myoblasts were silenced using DEGS1 CRISPR-Cas9 genome editing. Unlike silencing of Sptlc1 and Cers2, Degs1 silencing led to reduced myogenesis, as demonstrated by reduced myotube fusion index, area, and muscle related gene expression (FIG. 5o-r).

In humans, in the Helsinki Birth Cohort Study, it was demonstrated that the allele of a SNP associated with reduced mRNA expression of DEGS1 in GTEx skeletal muscle expression database, was associated with impaired measures of senior fitness, including a lower distance walked in a 6-walking test, and worse flexibility, as measured by sit and reach. This findings are in contrast to observations with Sptlc1 and Cers2 silencing which lead to enhanced myogenesis.

These findings demonstrate that dihydroceramide accumulation through DEGS1 inhibition impairs muscle growth and age-related muscular fitness.

### EXAMPLE 8—SPHINGOLIPID DEPLETION IN MDX MICE ALLEVIATES DUCHENNE MUSCULAR DYSTROPHY

Using muscle biopsies from humans with muscular dystrophies upregulation of transcripts of the sphingolipid de novo biosynthetic pathways in patients with muscular dystrophies was observed, including SPTLC1, SPTLC2, KDSR, CERS2, CERS5, CERS6, and DEGS1 (FIG. 8a). The most significant upregulation was observed for Duchenne muscular dystrophy.

Next the activity of sphingolipid synthesis was inhibited in a mouse model of Duchenne muscular dystrophy. Mdx, a dystrophin deficient mouse model, was treated with myriocin (0.4 mg/kg/3 times a week intraperitoneally) for 2 months. After myriocin treatment, these mice displayed improved performance in uphill running test (FIG. 8b-c), improved grip strength (FIG. 8d), and improved performance in rotarod test (FIG. 8e). Furthermore, after downhill running, plasma levels of creatine kinase (CK) were lower in myriocin treated mice compared to controls (FIG. 8f). These findings demonstrate that inhibition of sphingolipid synthesis protects against muscular dystrophy.

### EXAMPLE 9—SPHINGOLIPID DEPLETION ALLEVIATES FRAILTY SYNDROME

Human frailty syndrome is characterized by weakness, decreased walking speed, exhaustion, weight loss, and low physical activity. Myriocin treated mice displayed reduced weakness, as demonstrated by increased grip strength (FIG. 2l), increased ad libitum walking speed (FIG. 9b), reduced exhaustion, as demonstrated by improved performance on a treadmill and increased maximal oxygen consumption (FIG. 2j-k), higher lean body as well as muscle mass (FIG. 2c-d), and higher physical activity, as demonstrated by longer distance travelled ad libitum during 10 min (FIG. 9a). Frailty predicts falls in older people. Myriocin treated mice reached a higher speed before they fell off the rotarod (FIG. 2m), as well as were faster in crossing a beam (FIG. 2n), demonstrating their improved coordination. In object recognition test, myriocin treated mice spent more exploring both the familiar object and novel object as compared to their DMSO treated counterparts (FIG. 9d). Although this could be partly attributable to the higher physical activity of myriocin treated mice, the treated mice were more interested in the novel than familiar object as compared to untreated mice. Thus, sphingolipid synthesis inhibition improves memory, demonstrated by the higher recognition index (FIG. 9e). Collectively, our findings demonstrate that myriocin treatment prevents both physical and cognitive frailty in aged mice.

Frailty is associated with multimorbidity, including diabetes. Aged myriocin treated mice demonstrated improved glucose tolerance in an oral glucose tolerance test (FIG. 9c). Importantly, the myriocin treated were chow-fed, demonstrating that rather than high-fat diet induced insulin resistance, myriocin protect against age-related glucose intolerance. Our findings demonstrate inhibition of sphingolipid synthesis protects against age-related chronic diseases, and targeting of sphingolipid synthesis pathway could protect against age-related multimorbidity and reduce polypharmacy.

### EXAMPLE 10—INHIBITION OF CERAMIDE SYNTHASE 1 IMPROVES GRIP STRENGTH IN AGING

Inhibition of ceramide synthase 1 producing 18:0 sphingolipids by P053, an oral inhibitor of CERS1, has recently been shown to produce beneficial metabolic effects (Turner et. al., 2018). However, its role in age-related sarcopenia has not been explored.

Mice were treated with P053 for 1 month 3.6 mg/kg/day in food (chow diet). Then grip strength was measured. After 1 month of treatment, P053 treated mice displayed improved grip strength, suggesting that inhibition of CERS1 could attenuate frailty and sarcopenia (FIG. 15). These findings demonstrate that inhibition of other enzymes of the sphingolipid pathway could provide beneficial health outcomes, and the suitability of sphingolipid pathway for pharmacological approaches in general.

### EXAMPLE 11—PROTEOSTASIS

Mitochondrial dysfunction and collapse of cellular protein homeostasis (proteostasis) are hallmarks of several neuromuscular disorders, such as aging, and dystrophies (Wattin et al. (2018), Int J Mol Sci.; 19(1):178). Inclusion body myositis (IBM) is a muscle disease featuring impaired proteostasis, in addition to inflammation (Weihl and Pestronk (2010), Curr Opin Neurol.; 23(5):482-488).

Cell lines from IBM patients featuring impaired proteostasis were obtained. This was validated using Proteostat staining which targets general disruption in proteostasis. Myriocin treatment improved the phenotype of IBM cell lines, thus clearing protein aggregates from these cells (FIG. 16).

Myriocin also promoted proteostasis in the skeletal muscle of aged mice (Figure FIG. 17). The A11 staining, indicating increased deposition of oligomers in the formation of Amyloid beta, shows that myriocin cleared amyloid beta aggregates in these mice.

Together with improved clearance of amyloids from IBM cells treated with myriocin, these data demonstrate that inhibition of sphingolipid pathway improves proteostasis and could be used as a therapeutic strategy for proteostatic diseases.

### EXAMPLE 12—MATERIAL AND METHODS

In Vivo Studies

Animals. Young (2-month-old) and aged (18-month-old) C57Bl/6JRj mice were purchased from Janvier Labs. EchoMRI measuring the fat and lean body mass, were measured before and after the treatment. The dose of myriocin was 0.4 mg/kg/3 times a week. Myriocin (Enzo Life Sciences, Farmingdale, N.Y.) was first dissolved in DMSO which was then mixed with PBS so that each mouse received 1.5 μL DMSO per injection. Animals were fed on a standard chow diet. All animals were housed in micro-isolator cages in a room illuminated from 7:00 AM to 7:00 PM with ad libitum access to diet and water. All the animal experiments are authorized by animal license 2890.1 and 3341 in Canton of Vaud, Switzerland.

Measurement of sphingolipids. Plasma (100 μL) and weighed tissue samples (20-60 mg) were transferred to 2 ml Safe-Lock PP-tubes, and extracted. In brief, samples were homogenized using two 6 mm steal beads on a Mixer Mill (Retsch, Haan, GER; 2×10 sec, frequency 30/s) in 700 μL MTBE/MeOH (3/1, v/v) containing 500 μmol butylated hydroxytoluene, 1% acetic acid, and 200 μmol of internal standards (IS, d18:1/17:0 ceramide, d18:1/17:0 sphingomyelin, Avanti Polar Lipids, Alabaster, Ala., USA) per sample. Total lipid extraction was performed under constant shaking for 30 min at RT. After addition of 140 μL dH2O and further incubation for 30 min on RT, samples were centrifuged at 1,000×g for 15 min to establish phase separation. 500 μL of the upper, organic phase were collected and dried under a stream of nitrogen. Lipids were resolved in 700 μL 2-propanol/methanol/water (7/2.5/1, v/v/v) for UPLC-MS analysis. Remaining tissues were dried, solubilized in NaOH (0.3 N) at 65° C. for 4 h and the protein content was determined using Pierce™ BCA reagent (Thermo Fisher Scientific, Waltham, Mass., USA) and BSA as standard.

Chromatographic separation was modified after Knittelfelder et al. using an ACQUITY-UPLC system (Waters Corporation), equipped with a Luna omega C18 column (2.1×50 mm, 1.6 μm; Phenomenex) starting a 20 min linear gradient with 80% solvent A (MeOH/H2O, 1/1, v/v; 10 mM ammonium acetate, 0.1% formic acid, 8 μM phosphoric acid). The column compartment was kept on 50° C. A EVOQ Elite™ triple quadrupole mass spectrometer (Bruker) equipped with an ESI source was used for detection of lipids in positive ionization mode. Lipid species were analyzed by selected reaction monitoring (ceramide, [M+H]+ to m/z 264.3, 22 eV, 60 ms; sphingomyelin, [M+H]+ to m/z 184.1, 20 eV, 40 ms; resolution 0.7 Q1/Q3). Data acquisition was done by MS Workstation (Bruker). Data were normalized for recovery, extraction-, and ionization efficacy by calculating analyte/IS ratios (AU) and expressed as AU/g tissue or AU/mL plasma.

Measurement of deoxysphingolipids. Plasma and muscle sphingolipids were processed using a method adapted from. Briefly, 10-15 mg of muscle sample was extracted with 500 μL of −20° C. methanol, 400 μL of ice-cold saline, 100 μL of ice-cold water and spiked with internal standard deoxysphinganine d3 (Avanti lipids). An aliquot (50 μL) of homogenate was dried under air and resuspended in RIPA buffer for protein quantification using BCA assay (BCA Protein Assay, Lambda, Biotech Inc., US). To the remaining homogenate, 1 mL of chloroform was added and the samples were vortexed for 5 min followed by centrifugation at 4° C. for 5 min at 15 000×G. The organic phase was collected and 2 μL of formic acid was added to the remaining polar phase, which was re-extracted with 1 mL of chloroform. Combined organic phases were dried and the pellet was resuspended in 500 μL of methanol and subsequent extraction steps were identical as described for plasma.

Fifty microliters of plasma was mixed with 500 μL of methanol and spiked with internal standard of deoxysphinganine d3 (Avanti lipids). The samples were placed on a mixer for 1 h at 37° C., centrifuged at 2800×G and the supernatant collected and acid hydrolyzed overnight at 65° C. with 75 μL of methanolic HCl (1N HCl, 10M H2O in methanol). Next, 100 μL of 10 M KOH was added to neutralize. 625 μL of chloroform, 100 μL of 2N NH4OH and 500 μL of alkaline water were added, the sample vortex-mixed and centrifuged for 5 min at 16 000 g. The lower organic phase was washed three times with alkaline water and dried under air. LCMS analysis was performed on an Agilent 6460 QQQ LC-MS/MS. Metabolite separation was achieved with a C18 column (Luna 100×2.1 mm, 3 μm, Phenomenex). Mobile phase A was composed of a 60:40 ratio of methanol:water and mobile phase B consisted of 100% methanol with 0.1% formic acid with 5 mM ammonium formate added to both mobile phases. The gradient elution program consisted of holding at 40% B for 0.5 min, linearly increasing to 100% B over 15 min, and maintaining it for 9 min, followed by re-equilibration to the initial condition for 10 min. The capillary voltage was set to 3.5 kV, the drying gas temperature was 350° C., the drying gas flow rate was 10 L/min, and the nebulizer pressure was 60 psi. Sphingoid bases were analyzed by SRM of the transition from precursor to product ions at associated optimized collision energies and fragmentor voltages (Table below). Sphingoid bases were then quantified from spiked internal standards of known concentration.

| Sphingoid base | Parent ion | Daughter ion | N |
|---|---|---|---|
| m17:0 doxSA | 272.4 | 254.4 | 13 |
| m18:0 doxSA | 286.3 | 268.4 | 13 |
| m18:0 doxSA d3 | 289.3 | 271.5 | 13 |

Endurance running test. The exercise regimen on a treadmill commenced at a speed of 9 cm/s. As the mice were aged, an inclination of 0 degrees was used. Mice were considered to be exhausted, and removed from the treadmill if they received 7 or more shocks (0.2 mA) per minute for two consecutive minutes. The distance traveled and time before exhaustion were registered as maximal running distance and time.

Grip strength. Muscle strength was assessed by grip strength test. The grip strength of each mouse was measured on a pulldown grid assembly connected to a grip strength meter (Columbus Instruments). The mouse was drawn along a straight line parallel to the grip, providing peak force. The experiment was repeated three times, and the highest value was included in the analysis.

Rotarod test. The rotarod test measures muscle strength, coordination, and endurance. Mice were left undisturbed in the room for 30 min. The speed of the rotating cylinder (rotarod) increased from 0 to 40 rpm in 5 min. Each mouse had 3 trials per day for 3 consecutive days. The latency and speed the mouse reached it passive rotation or fall from the rotor was recorded, and the latency and speed of the best trial of the second day is presented.

Crossbar test. Crossbar test assesses active balance through the ability to balance while walking along an elevated beam to reach a dark end side where they are able hide. Since all the mice were easily able to cross a squared (3 cm) beam, and a circular beam of diameter 1.5 cm was too difficult to them, the latency was recorded to cross a circular beam of 3 cm. The mice were trained for one day before the actual trial was recorded, and the average of the latency of three trials per mouse is presented.

Stem cell isolation. Gastrocnemius, soleus, and quadriceps muscles from both hindlimbs were excised and transferred into PBS on ice. All muscles were trimmed, minced and digested with 2.5 U/ml of Dispase II (Roche) and 0.2% Collagenase B (Roche) in PBS for 30 mins at 37° C. Samples were then centrifuged at 50 g for 5 min followed by removing the supernatant and further digested for 20 mins at 37° C. twice. Muscle slurries were sequentially filtered through 100 μm and 40 μm cell strainers. The isolated cells were then washed in washing buffer (PBS+2.5% BSA) and resuspended in 800 μL of washing buffer. They were immediately stained with antibodies, including CD31 (1:800, eBioscience, eFluor450 conjugated); CD45 (1:200, eBioscience, eFluor450 conjugated); Sca-1 (1:1000, eBioscience, PE-Cy7 conjugated); CD11b (1:100, eBioscience, eFluor450) and CD34 (1:100, BD Pharmingen, FITC conjugated); alpha-7 integrin (1:50, RD system, eFluor700 conjugated) for 45 min at 4° C. Secondary staining was performed with propidium iodide (PI, Sigma) for 15 min at 4° C. in the dark. Stained cells were analyzed and sorted using the FACSAria II instrument (BD Biosciences). Debris and dead cells were excluded by forward scatter, side scatter and PI gating.

Cardiotoxin-induced muscle damage and MuSC transplantation. MuSCs were transplanted from donor mice to recipient mice. The donor mice were aged male C57Bl/6JRj mice, whose treatment with myriocin was started at the age of 18 months. The recipient mice were either aged (18-month-old) male C57Bl/6JRj mice or 1-year-old male mdx mice. 50 μL of 20 μM Naje Mossambica mossambica cardiotoxin (Sigma) was injected intramuscularly into the tibialis anterior (TA) muscle of recipient mice before transplantation. 24 h after CTX injection, equal number (1500) of freshly isolated MuSCs from donor mice were injected intramuscularly into the TA muscle. Recipient mice were sacrificed at 7 and 14 days after transplantation.

Histology. TA muscles were harvested from anesthetized mice, and immediately embedded in Thermo Scientific™ Shandon™ Cryomatrix™ and frozen in isopentane, cooled in liquid nitrogen, for 1 min before being transferred to dry ice and stored at −80° C. 8 μm cryosections were incubated in 4% PFA for 15 min, washed three times for 10 min with PBS, incubated for antigen retrieval in pH 6.0 citrate buffer for 10 min at 95° C. (for PAX7 antibody), counterstained with DAPI, laminin (1:200, Sigma), PAX7 (1:200, DSHB, University of Iowa), dystrophin (1:100, Spring Bioscience) or eMyHC (1:50, DSHB, University of Iowa), coupled with Alexa-488 or Alexa-568 fluorochromes (Life Technology) and mounted with Dako Mounting Medium. Microscopy images of fluorescence from muscle fibers were analyzed using the ImageJ software. Centralized nuclei percent, minimal Feret diameter and cross-sectional area in TA muscles were determined using the ImageJ software quantification of laminin, dystrophin, and DAPI-stained muscle images from VS120-S6-W slides scanner (Olympus). A minimum of 2,000 fibers were used for each condition and measurement. The minimal Feret diameter is defined as the minimum distance between two parallel tangents at opposing borders of the muscle fiber. This measure has been found to be resistant to deviations away from the optimal cross-sectioning profile during the sectioning process. The mean cross-sectional area of muscle fibers was calculated as the average cross-sectional area of 2,000 fibers per from sample. 7-8 mouse samples per condition were used for histological quantification of donor mice. The eMyHC quantification is expressed as proportion of eMyHC positive signal over total TA cross sectional area. Following MuSC injection in CTX injected recipient mice, inflammation representing the regenerative stage of muscle was quantified by ImageJ software as proportion of inflammatory area over total area of the TA muscle cross section. The PAX7-positive cells were quantified as the average number of cells per field of more than 30 randomly chosen fields from a mouse TA. For each quantification, 3 or more mice were used.

Ex vivo analysis of MuSCs. MuSCs were isolated as described above, and seeded in 96- or 48-well plates. The cells were incubated in 30 μM myriocin containing medium (Ham's F-10 nutrient mixture, FBS 20%, basic fibroblast growth factor 2.5 ng/mL, penicillin 100 U/mL, streptomycin 100 μg/mL) for 72 hours. PFA 4% was applied for 15 min, the cells were washed three times for 10 min with PBS, and were blocked in 2% BSA in PBS. The cells were then incubated in primary antibodies MYOD (1:50, LabForce) and MYOG (1:50, Santa Cruz) overnight at 4° C. Secondary antibodies were coupled with Alexa-488 or Alexa-568 fluorochromes (Life Technology) and mounted with Dako Mounting Medium. Leica DMI 4000 microscope was used to image the cells. Quantification of the MYOD+ and MYOG+ cell number was based on more than 500 cells per condition.

Novel Object Recognition. The novel object recognition task is used to assess recognition memory of mice. It is based on the natural tendency of rodents to explore a novel object by comparison to a familiar one. A habituation phase is realized to familiarize animals to the arena context. 24 hours later, during the acquisition phase (learning phase), two identical objects are placed in the arena and presented to the mouse. During the retention phase (3 hours following the acquisition phase), one of the two familiar objects is replaced by a novel one. Measures of duration and number of exploration for each object are realized, during both acquisition and retention phases, and a Recognition Index is calculated.

c. Severity Grade

Grade 0

In Vitro Studies

Cell culture and cell transfection. The C2C12 mouse myoblast cell line was obtained from the American Type Culture Collection (CRL-1772™). C2C12 cells or clones were cultured in growth medium consisting of Dulbecco's modified Eagle's medium (Gibco, 41966-029), 20% Fetal Bovine Serum (Gibco, 10270-106) and 100 U/mL penicillin and 100 mg/mL streptomycin (Gibco, 15140-122). To induce differentiation, FBS was substituted with 2% horse serum (Gibco, 16050-122). Trypsin-EDTA 0.05% (GIBCO, 25300-062) was used to detach cells. Human skeletal muscle cells were obtained from Lonza (SkMC, #CC-2561) and cultured in growth medium consisting of DMEM/F12 (Gibco, 10565018), 20% Fetal Bovine Serum (Gibco, 10270-106) and 100 U/mL penicillin and 100 mg/mL streptomycin (Gibco, 15140-122). To induce differentiation, FBS was reduced to 2% and kept in culture. All cells were maintained at 37° C. with 5% CO2. Cell transfections were done using TransiT (Mirus) according to the manufacturer's protocol with a 3:1 ratio of transfection agent to DNA. C2C12 cells were grown confluent, and 30 μM myriocin or DMSO was added and cells were kept in growth medium for another 3 days. Sptlc1 clones were plated to reach confluency simultaneously and were kept in growth medium for 3 days before using them for immunocytochemistry or RNA isolation. The concentration of myriocin in medium for all experiments was 30 μM. A stock solution of 20 mM myriocin in DMSO was used dissolve myriocin, and a corresponding volume of DMSO without myriocin was used as control.

CRISPR guide RNA design and cloning. Two guide RNAs per gene were designed with the help of the online GPP web portal tool (https://portals.broadinstitute.org/gpp/public/analysis-tools/sgrna-design) using Streptococcus pyogenes PAM sequence (NGG). The guide RNAs with best predicted on- and off-target scores were selected. The guide RNA sequences are listed in Table 2. The oligonucleotides were synthesized (Microsynth, Switzerland) and cloned into the CRISPRv2 plasmid (addgene #52961) using the BsmBI restriction sides. The insertion by cloning was verified by Sanger sequencing (Microsynth, Switzerland). To test the efficiency of the guide RNAs, the cloned vectors were transiently transfected (TransIT, Mirus) in C2C12 cells, and 48 h after transfection RNA was isolated, reverse transcribed and gene expression was measured by RT-qPCR.

Creation of single clone Sptlc1 knockout in C2C12 myoblasts. C2C12 cells were transfected with the lentiCRISPR v2 plasmid containing the gRNAs targeting exon 1 of Sptlc1 or the empty vector lentiCRISPR v2 plasmid as a control. 36 h after transfection cells were selected with 2 μg/mL puromycin (Invivo gene; QLL3803A) for 3 d and single cell sorted. Five to ten different clones for each gRNA were grown without selection marker. DNA from these clones was isolated (Macherey-Nagel, 740952) and PCR amplified (see Table 2). The PCR product was gel purified (Machery-Nagel, 740609) and Sanger sequencing was performed to verify the clones with deletions or insertions. In one of the clones, Sptlc1 gRNA1 led to a homozygous knockout of Sptlc1 (Sptlc1−/−) whereas gRNA2 led to a heterozygous knockout of Sptlc1 (Sptlc+/−) in another clone.

Creation of polyclonal Sptlc1 or Cers2 knockouts in C2C12 myoblasts. Lentivirus were produced from lentiCRISPRv2 plasmids containing no gRNA (empty vector), Sptlc1 gRNA2 or Cers2 gRNA2 (Table 3) by co-transfection with the packaging of plasmids pMD2G and psPAX2 in HEK 293T cells using lipofectamine 2000. Viral supernatants were harvested 36 to 48 h post transfection. C2C12 were transduced with viral supernatant for 20 hours. 24 h later, cells were selected with 3 μg/mL puromycin for 3 days. Reduction in the target protein was confirmed by Western blotting.

Western blotting. C2C12 cells were lysed on ice in RIPA buffer composed of Tris HCl 50 mM, NaCl 5 M, EDTA 5 mM, SFS 0.1%, NAF 100 mM, sodium deoxycholate 5 mg/mL, and NP40 1% containing protease and phosphatase inhibitors (Roche). Protein concentrations were determined using Bradford method, and 23 μg of protein loaded on a 12% SDS-PAGE gel. After electrophoresis, proteins were separated by SDS-PAGE and transferred onto methanol activated polyvinylidene difluoride membranes. Blocking of the membranes was done in 5% milk-TBST for 1 h, and after wash, the membranes were incubated overnight with primary antibody anti-SPTLC1 (Proteintech) 5% BSA-TBST 1:1000 or anti-CERS2 (Sigma) in 3% BSA-TBST 1:1000. Incubation with secondary anti-rabbit polyclonal antibody was done in 5% BSA-TBST 1:2000. Antibody detection reactions were developed by enhanced chemiluminescence (Advansta), and imaged using the c300 imaging system (Azure Biosystems).

Myoblast proliferation assay in vitro. To measure cell proliferation in CRISPR mediated Sptlc1 knockout or myriocin-treated cells, 3000 cells per well were seeded in 96-well plates (Greiner bio-one, CELLSTAR, 655180), cultured in growth medium and either transfected (TransIT, Mirus) 24 h post seeding with 30 ng lentiCRISPR v2 plasmid (addgene #52961) containing a guide RNA (Table 2) or treated with 30 μM myriocin diluted in DMSO, or DMSO as control. Cell proliferation was measured according to the manufacturer's protocol (Cell proliferation ELISA BrdU, Roche). In brief, proliferating myoblasts were labelled with 10 μL/well BrdU for 2 h at 37° C., fixed with 200 μL/well for 30 min at room temperature and incubated with the supplied BrdU antibody for 90 min at room temperature. After washing three times with PBS, cells were incubated in 100 μL/well substrate solution for 10 min before measuring 370 nm absorbance using the ELISA plate reader (Perkin Elmer, Victor™ X4).

RNA isolation and real time qPCR. RNA was isolated using the RNeasy Mini kit (Qiagen, 74106) and reverse transcribed with the High-Capacity-RNA-to-cDNA kit (Thermo Fisher, 4387406). Gene expression was measured by qPCR using the Power SybrGreen Master mix (Thermo Fisher, 4367659). All quantitative polymerase chain reaction (PCR) results were calculated relative to the mean of the housekeeping gene Gapdh. The average of two technical replicates was used for each biological data point. Primer sets for quantitative reverse transcription PCR (q-RT-PCR) analyses are shown in Table 1.

Immunocytochemistry. C2C12 cells cultured on a sterilized cover slip in 6-well plates (Greiner bio-one, CELLSTAR, 657160) were fixed in Fixx solution (Thermo Scientific, 9990244) for 15 min and permeabilized in 0.1% Triton X-100 (Amresco, 0694) solution for 15 min at room temperature. Cells were blocked in 3% BSA for 1 h at room temperature to avoid unspecific antibody binding and then incubated with primary antibody over night at 4° C. with gentle shaking. MyHC was stained using the MF20 primary antibody (1:200, Invitrogen, 14-6503-82) for C2C12 cells and in Lonza muscle cells with a MYL2 antibody (1:140, Abcam, ab79935). The next day cells were incubated with secondary antibody (Thermo Fisher #A10037 for MF20 and #A-21206 for MYL2) for 1 h at room temperature and nuclei were labelled with DAPI. The immunofluorescence images were acquired using either fluorescence or confocal microscopy. The myofusion index was calculated as the ratio of nuclei within myotubes to total nuclei. The myotube diameter was measured for 8 myotubes per image using ImageJ. Myotube area was calculated as the total area covered by myotubes.

Gene expression and phenotype analysis in BXD mouse population. Quadriceps microarray data (Affymetrix Mouse Gene 1.0 ST) and phenotype data from BXD mouse genetic reference population were analyzed for Pearson correlations with R software. The first principal component of the ceramide biosynthetic pathway representing its expression in muscle was calculated by including the following genes: SPTLC1, SPTLC2, KDSR, CERS1, CERS2, CERS3, CERS4, CERS5, CERS6, DEGS1.

Quadriceps transcript profiling using RNA sequencing. Quadriceps muscles were collected and snap-frozen in liquid nitrogen from C57Bl/6JRj mice undergoing intraperitoneal myriocin treatment for 10 weeks on a chow diet starting at the age of 18 months. RNA was isolated using Direct-zol RNA kit (Zymo, Irvine, Calif.). RNA quality was assessed using Agilent 2100 BioAnalyzer (Agilent, Santa Clara, Calif.). Samples with RIN≥8, 28S/18S≥1.0, and c≥20 ng/μL were included in analyses. Using these criteria, 9 (4 DMSO and 5 myriocin) samples passed and 2 samples failed to pass quality control criteria. Sequencing libraries were prepared by BGI genomics using the DNBsea™ technology. Paired end sequencing with 100 cycles was performed using the BGISEQ-500 instrument. After removal of adaptor sequences and low quality reads, at least 56 million reads per sample was obtained. SOAPnuke was used to obtain clean reads (parameters -l 15, -q 0.2, -n 0.05). Reads were mapped using STAR aligner version 2.5.2b using the mouse GRCm38 genome assembly and the release 91 GTF annotation from Ensembl. Htseq-count version 0.6.0 was used to count the number of reads mapping to genes (mode=union, type=exon, idattr=gene_id). Transcript displaying higher expression than log 2(CPM+1)>0.5 in at least 3 samples were included in analyses. Differential gene expression analysis and expression normalization was performed using voom, ‘variance modeling at the observational level’, adjusting for sacrifice date. At individual gene level, no gene passed the multiple testing correction threshold. Benjamini-Hochberg correction for multiple testing was used. For gene set enrichment analysis using gene ontology (GO) categories, transcripts were ordered according to their log 2-transformed fold change, and 100,000 permutations were used. Adjusted p-value<0.05 was considered significant.

Human Studies

Young vs. old skeletal muscle microarrays: Gene expression analysis of young vs. old human muscle biopsies was obtained from publicly available dataset GSE25941. Briefly, a total of 36 subjects were included in the study. The young (n=15, 25±1 y) participants included 7 males and 8 females. The old (n=21, 78±1 y) participants included 10 males and 11 females. All subjects were healthy and had never been involved in any formal exercise. Skeletal muscle biopsies were obtained from the vastus lateralis in the basal state. Affymetrix Human Genome U133 Plus 2.0 Array platform was used to perform the microarray analysis.

Gene expression analysis from human skeletal muscle in Genotype-Tissue Expression (GTEx) project. For RNA gene expression analyses, 491 post-mortem skeletal muscle biopsies from the GTEx gene expression collected were employed. As measures of gene expression, residual expression levels of transcripts adjusting for the published GTEx v7 covariates was used. As for eQTL analyses, the GTEx v7 genotypes (dbGAP, approved request #10143-AgingX) was used. For the combined expression of SPTLC1 and SPTLC2, mean of the residual expression was used.

Helsinki Birth Cohort Study (HBCS): The HBCS includes 13,435 individuals born in Helsinki between 1934 and 1944. The senior fitness test (SFT) describing the physical performance of the participants was performed to 695 individuals. Here, a modified test battery was used, consisting of five components of the SFT: number of full arm stands in 30 seconds with arms folded across chest to assess lower body strength; number of biceps curls in 30 seconds while holding a hand weight (3 kg for men and 2 kg for women) to assess upper body strength; chair sit and reach to assess the lower body flexibility (from sitting position with leg extended at front of chair and hands reaching toward toes, number of cm (plus/minus) from extended fingers to tip of toe); number of meters walked in 6 minutes to measure aerobic endurance; and back scratch to assess upper body flexibility (with one hand reaching over shoulder and the other one up middle of back, distance (in cm) between extended middle fingers (plus/minus). The result of each test was expressed as age (for each 5-year group) and sex-standardized percentile scores. An overall test score was calculated by summarizing the normalized scores of the 5 SFT components. Isometric grip strength of the dominating hand was tested by a Newtest Grip Force dynamometer (Newtest Oy, Oulu, Finland). The maximum value of three squeezes was used in analyses.

DNA was extracted from blood samples and genotyping was performed with the modified Illumina 610k chip by the Wellcome Trust Sanger Institute, Cambridge, UK, according to standard protocols. Genomic coverage was extended by imputation using the 1000 Genomes Phase I integrated variant set (v3/April 2012; NCBI build 37/hg19) as the reference sample and IMPUTE2 software. Before imputation the following QC filters were applied: SNP clustering probability for each genotype >95%, Call rate >95% individuals and markers (99% for markers with MAF<5%), MAF>1%, HWE P>1×10-6. Moreover, heterozygosity, gender check and relatedness checks were performed and any discrepancies removed.

For identification of cis-eQTLs eQTLs±1 Mb were analyzed from the start and the end of SPTLC1 and SPTLC2. SNPs with minor allele frequency >10% were included. SNPs with r2>0.2 were incorporated in the same haploblock. As there were 4 haploblocks within both SPTLC1 and SPTLC2 region (300 kb), Bonferroni correction was used for 25 haploblocks (P<0.002) within the 2 Mb region studied for identification of cis-eQTLs. Linear regressions were performed with SNPtest assuming an additive genetic model. All models were adjusted for age, sex, highest education achieved (basic or less/upper secondary/lower tertiary/upper tertiary) and smoking (yes/no).

1. An inhibitor of sphingolipid adapted for use in treatment or prevention of a muscle disease.

2. The inhibitor of sphingolipid for use of claim 1, wherein the muscle disease comprises frailty comprising sarcopenia and/or muscle atrophy, and/or is sarcopenia and optionally is frailty comprising sarcopenia and/or muscle atrophy.

3. The inhibitor of sphingolipid for use of claim 2, wherein the frailty comprises
(i) sarcopenia and/or muscle atrophy, and
(ii) cognitive impairment.

4. The inhibitor of sphingolipid for use of claim 3, wherein the cognitive impairment is senile dementia.

5. The inhibitor of sphingolipid for use of claim 1, wherein the sphingolipid comprises sphinganines, sphingosines, ceramides, dihydroceramides, sphingomyelins, deoxysphingolipids optionally 1-deoxysphinganine) or any combination thereof.

6. The inhibitor of sphingolipid for use of claim 1, wherein the inhibitor is an inhibitor of one or more enzymes involved in biosynthesis of sphingolipid.

7. The inhibitor of sphingolipid for use of claim 6, wherein the one or more enzymes is or are selected from SPTLC1, SPTLC2, SPTLC3, KDSR, CERS1, CERS2, CERS3, CERS4, CERS5, CERS6, SGMS1, SGMS2, SMPD1, SMPD2, SMPD3, SMPD4, ASAH1, ASAH2, ASAH2B, ASAH2C, ACER1, ACER2, ACER3, SPHK1 and SGPP1.

8. The inhibitor of sphingolipid for use of claim 6, wherein the inhibitor inhibits
(i) expression of a nucleic acid molecule encoding one or more enzymes being involved in biosynthesis of sphingolipid, or
(ii) enzymatic activity of an enzyme being involved in biosynthesis of sphingolipid.

9. The inhibitor of sphingolipid for use of claim 8, wherein
(I) the inhibitor of (i) is selected from a small molecule, an aptamer, a siRNA, a shRNA, a miRNA, a ribozyme, an antisense nucleic acid molecule, a CRISPR-Cas9-based construct, a CRISPR-Cpf1-based construct, a meganuclease, a zinc finger nuclease, and a transcription activator-like (TAL) effector (TALE) nuclease, and/or
(II) the inhibitor of (ii) is selected from a small molecule, an antibody or antibody mimetic, and an aptamer.

10. The inhibitor of sphingolipid for use of claim 9, wherein the antibody mimetic is selected from affibodies, adnectins, anticalins, DARPins, avimers, nanofitins, affilins, Kunitz domain peptides, Fynomers®, trispecific binding molecules and probodies.

11. The inhibitor of sphingolipid for use of claim 9, wherein the small molecule of (ii) is myriocin or a small molecule inhibitor selected from Table A, or a salt or ester thereof.

12. The inhibitor of sphingolipid for use of claim 6, wherein the inhibitor edits a genome at a location encoding an enzyme being involved in biosynthesis of sphingolipid.

13. The inhibitor of sphingolipid for use of claim 1, wherein the muscle disease is inclusion body myositis or a muscular dystrophy, optionally Duchenne muscular dystrophy.

14. A method for treating and/or preventing muscle disease comprising administering an inhibitor of sphingolipid to a subject in need thereof.
